(19) United States
(12) Patent Application Publication
(10) Pub. No.: US 2010/0286644 A1
(43) Pub. Date: Nov. 11, 2010

(54) Substrates With Printed Patterns Thereon Providing A Three-Dimensional Appearance

(76) Inventors: Wenbin Li, Mason, OH (US); George Christopher Dobrin, Mason, OH (US); Ada Ho Yau Ko, Cincinnati, OH (US); Douglas Joseph Meyer, Cincinnati, OH (US); Alrick Vincent Warner, Loveland, OH (US); Kathleen Marie Lawson, West Chester, OH (US); Jonathan Aaron Lu, Cincinnati, OH (US); Angelli Sue Denmon, Hamilton, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/844,022

(22) Filed: Jul. 27, 2010

Related U.S. Application Data
(63) Continuation of application No. 11/946,969, filed on Nov. 29, 2007.
(60) Provisional application No. 60/861,910, filed on Nov. 29, 2006.

(51) Int. Cl. A61F 13/15

(52) U.S. Cl. 604/385.01

(57) ABSTRACT

Aspects of the present disclosure involve patterns on substrate surfaces, such as nonwoven webs or fabrics, plastic films, and laminates thereof, that cause the substrate surfaces to exhibit a three-dimensional appearance. In some embodiments, the three-dimensional appearance of the substrate surface resembles protrusions and indentions indicative of threads in woven cloths. The patterns are created by printing a surface of a substrate, as opposed to deforming the substrate such as by embossing. Embodiments of the patterns include a plurality of repeating shapes or macro-units disposed on the substrate surface.

### CROSS REFERENCE TO RELATED APPLICATIONS

This application is a continuation of U.S. application Ser. No. 11/946,969, filed on Nov. 29, 2007, which claims the benefit of U.S. Provisional Application 60/861,910, filed Nov. 29, 2006, which are both hereby incorporated by reference.

### FIELD OF THE INVENTION

The present disclosure relates to substrates such as films and fabrics, and more particularly, to films and fabrics with printed patterns thereon to provide a three-dimensional appearance.

### BACKGROUND OF THE INVENTION

Substrates, such as nonwoven webs or fabrics, plastic films, and the like are known in the art and have various properties, such as strength and fluid handling characteristics, that make them useful in many products, such as consumer goods (e.g. absorbent articles), commercial goods (e.g. medical products), and packages for such goods. In one example, absorbent articles such as diapers and incontinent briefs worn by infants and other incontinent individuals are configured to receive and contain discharged urine and other body exudates. These articles may be constructed with numerous layers of substrates such as nonwoven and woven fabrics and/or plastic films. More particularly, such absorbent articles may include a chassis having an inner, body-facing topsheet and an outer, garment-facing backsheet with an absorbent core disposed in between. The topsheets and/or backsheets of such articles are sometimes constructed from nonwoven webs, plastic films, and/or laminates thereof. The topsheets and backsheets of such absorbent articles may function to absorb and/or contain the discharged materials and also to isolate bodily exudates from the wearer's skin and from the wearer's garments and bed clothing. It is typical for these substrates to be substantially smooth, flat and aesthetically unappealing. Efforts have been made to modify these substrates in order to provide them with a particular appearance. For example, such substrates may be modified to exhibit a softer, quilted, and/or cloth-like appearance. For instance, it may be desirable to provide a diaper having a backsheet which may include a film/nonwoven laminate with a cloth-like appearance. Knitted or woven clothes have a three-dimensional appearance that is readily noticeable by a person. As such, nonwoven fabrics and/or plastic films are sometimes modified to provide a physical or actual three-dimensional pattern which gives a more cloth-like appearance to the visible surface of the laminate. Non-limiting examples of known methods which provide an actual three-dimensional appearance to a substrate include embossing and hydro-molding. The physical modification of the substrate to provide an actual three-dimensional pattern also provides the substrate with a noticeable three-dimensional texture. Without intending to be bound by any theory, it is believed that a person such as a caregiver may notice the presence of an actual three-dimensional pattern or texture (that may be for example include peaks and valleys that are present on the surface of an embossed substrate) when he or she sees the bright and dark zones on the substrate's surface. Since peaks receive more light than the valleys, the peaks may appear to a person brighter than the valleys. In addition, the peaks may cast a shadow which tends to darken the valleys even further.

Although embossing or hydro-molding may provide the desired three-dimensional appearance to a substrate, there are disadvantages associated with such processes. Although a substrate may have at least partial plastic properties, embossing such a substrate may cause it to “shrink” in the sense that the formation of a three-dimensional pattern has to be somehow compensated by a reduction in size of the substrate. As a result, a greater amount of material may be needed for a particular use than would otherwise have been required with a flat material. In addition, embossing or hydromolding may also act to weaken the substrate in particular when the substrate which is embossed as a relatively low basis weight. As such, substrates with relative high basis weights may be required when embossing. Further, these processes oftentimes require the manufacturer to realize a significant investment in capital in order to acquire equipment such as embossing rollers or hydro-molding drums or belts. Such a significant investment in capital can make it cost prohibitive for a manufacturer to replace its equipment as often as it would want to and may also prevent a manufacturer to provide a large number of three-dimensional patterns on its substrate.

The literature is also replete with articles that include a substrate that is printed to display various graphics such as designs, characters, icons, and the like in order to make the article more aesthetically appealing. Such designs, characters, and icons may be printed to provide a three-dimensional appearance to the designs, characters, and icons themselves. However, the printing of these designs, characters, and/or icons on a substrate may not alter the appearance of substrate itself. As a result, a person looking at the substrate may not perceive and/or believe that the substrate itself is three-dimensional. Substrates which include an actual three-dimensional pattern or texture and which are printed to include a graphic are also known in the art.

As discussed in detail below, aspects of the present disclosure involve printing a substrate to provide the substrate with a perceived three-dimensional appearance without necessarily physically modifying the substrate itself.

### SUMMARY OF THE INVENTION

Aspects of the present disclosure involve printing a repeating pattern on a substrate such as a nonwoven web or fabric, plastic film, and laminate thereof in order to provide this substrate with a perceived three-dimensional pattern, which may cause a visible surface of the substrate to exhibit a three-dimensional appearance. In some embodiments, the three-dimensional appearance of the substrate surface resembles protrusions and indentions indicative of threads in woven cloths. The patterns are created by printing a surface of a substrate, as opposed to altering or deforming the substrate such as by embossing or hydro-molding.

In one form, a disposable absorbent article adapted to be worn about a lower torso region of a wearer includes: a chassis including a first waist region, a second waist region, a crotch region disposed intermediate the first waist region and the second waist region, and an absorbent core disposed in the crotch region, the chassis including a substrate; wherein the substrate comprises a sheet having a first surface and a second surface disposed opposite the first surface, the sheet including a repeating pattern of macro-units printed on the first surface; wherein the macro-units include a first color zone defining a L* value of L1, a second color zone defining a L* value of L2, and a third color zone defining a L* value of L3; and wherein L1>L2>L3, 3≦(L1−L3), and 2≦(L1−L2)≦10.

In another form, a disposable absorbent article adapted to be worn about a lower torso region of a wearer includes: a chassis including a first waist region, a second waist region, a crotch region disposed intermediate the first waist region and the second waist region, and an absorbent core disposed in the crotch region, the chassis including a substrate; wherein the substrate comprises a sheet having a first surface and a second surface disposed opposite the first surface, the sheet including a repeating pattern of macro-units printed on the first surface; wherein the macro-units include at least a first color zone defining a L* value of L1, a second color zone defining a L* value of L2, a third color zone defining a L* value of L3, and a fourth color zone defining a L* value of L4; and wherein L1>L2>L3>L4, 2≦(L1−L2)≦10, 2≦(L2−L3), and 2≦(L3−L4).

In yet another aspect, a substrate includes: a sheet having a first surface and a second surface disposed opposite the first surface; a repeating pattern of macro-units printed on the first surface; wherein the macro-units include a first color zone defining a L* value of L1, a second color zone defining a L* value of L2, and a third color zone defining a L* value of L3; and wherein L1>L2>L3, 3≦(L1−L3), and 2≦(L1−L2)≦10.

In still another aspect, a substrate includes: a sheet having a first surface and a second surface disposed opposite the first surface; a repeating pattern of macro-units disposed on the first surface; wherein the macro-units include at least a first color zone defining a L* value of L1, a second color zone defining a L* value of L2, a third color zone defining a L* value of L3, and a fourth color zone defining a L* value of L4; and wherein L1>L2>L3>L4, 2≦(L1−L2)≦10, 2≦(L2−L3), and 2≦(L3−L4)

### BRIEF DESCRIPTION OF THE DRAWINGS

FIG. 1 shows a top view of one embodiment of a repeating pattern printed on the surface of a substrate.

FIG. 2 is one embodiment of a macro-unit having four color zones.

FIG. 3 is an illustration of three axes (i.e. L*, a*, and b*) used with the CIELAB color scale.

FIG. 4 shows one example of how a pattern may be printed on a substrate.

FIG. 5 is a plan view of FIG. 5 looking in the cross direction.

FIG. 6 is a plan view of FIG. 5 looking in the machine direction.

FIG. 7 is detailed view of a single macro-unit from the pattern of FIG. 1.

FIG. 8 shows a plurality of generally circular-shaped macro-units having different sizes and different numbers of colored zones.

FIG. 9 shows a plurality of generally square-shaped macro-units having different sizes and different numbers of colored zones.

FIG. 10 illustrates a plurality of macro-units with print point rectangles used to estimate the distances between adjacent macro-units.

FIG. 11 illustrates an embodiment of a printed area of a substrate having an outer perimeter that defines a rectangular shape having four sides.

FIG. 12 illustrates an embodiment of a printed area of a substrate having an outer perimeter that defines a circular shape.

FIG. 13 illustrates an embodiment of a printed area of a substrate having an outer perimeter that defines a triangular shape.

FIG. 14 is a top plan view of a disposable incontinent absorbent article that may utilize one or more substrates having patterns disposed thereon in accordance with the present disclosure.

FIG. 15 shows a first example of a pattern that may be applied to various substrates.

FIG. 16 shows a second example of a pattern that may be applied to various substrates.

FIG. 17 shows a third example of a pattern that may be applied to various substrates.

FIG. 18 shows a fourth example of a pattern that may be applied to various substrates.

### DETAILED DESCRIPTION OF THE INVENTION

The following term explanations may be useful in understanding the present disclosure:

“Absorbent article” is used herein to refer to consumer products whose primary function is to absorb and retain soils and wastes.

“Absorbent article for inanimate surface” is used herein to refer to consumer products whose primary function is to absorb and retain soils and wastes that may be solid or liquid and which are removed from inanimate surfaces such as floors, objects, furniture and the like. Non-limiting examples of absorbent articles for inanimate surfaces include dusting sheets such as the SWIFFER cleaning sheets, pre-moistened wipes or pads such as the SWIFFER WET pre-moistened cloths, paper towels such as the BOUNTY paper towels, dryer sheets such as the BOUNCE dryer sheets and dry-cleaning clothes such as the DRYEL cleaning clothes all sold by The Procter & Gamble Company.

“Absorbent article for animate surface” is used herein to refer to consumer products whose primary function is to absorb and contain body exudates and, more specifically, refers to devices which are placed against or in proximity to the body of the user to absorb and contain the various exudates discharged from the body. Non-limiting examples of incontinent absorbent articles include diapers such as PAMPERS diapers, training and pull-on pants such as PAMPERS FEEL 'N LEARN and EASY UPS, adult incontinence briefs and undergarments such as ATTENDS adult incontinence garments, feminine hygiene garments such as panty liners, absorbent inserts, and the like such as ALWAYS and TAMPAX, toilet paper such as CHARMIN toilet paper, tissue paper such as PUFFS tissue paper, facial wipes or clothes such as OLAY DAILY FACIAL wipes or clothes, toilet training wipes such as KANDOO pre-moistened wipes, all sold by The Procter & Gamble Company.

“Consumer product” is used herein to refer to products that are manufactured and sold on a large industrial scale (i.e. hundreds of thousand of units), which is generally sold in packaged form and may be purchased by consumers from various retail stores.

The terms “actual size” or “actual dimension” are used herein to refer to the physical size of an object in at least one dimension, which is measured via any suitable means or tool known in the art and is expressed in meter, centimeter or millimeter.

The terms “perceived size” or “perceived dimension” are used herein to refer to the relative size of an object as it is perceived by a person having a 20-20 vision (normal or corrected) depending on the distance between the person and the object. For example, if two objects have the same actual size but are positioned at different distances from a person or viewer, the perceived size of the object which is closest to the viewer will be greater than the perceived dimension of the object which is farther away.

The term “diaper” is used herein to refer to an absorbent article generally worn by infants and incontinent persons about the lower torso.

The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

The term “disposed” is used herein to mean that an element(s) is formed (joined and positioned) in a particular place or position as a macro-unitary structure with other elements or as a separate element joined to another element.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “macro-unit” or “macro-cell” is used herein to describe an element on the surface of a substrate and whose overall shape is readily visible and/or noticeable by a person holding the substrate at a distance of about 30 cm from the person's eyes in natural daylight conditions. A macro-unit or cell may be formed of a plurality of micro-units whose overall shapes are not readily visible and/or noticeable by a person holding the substrate at a distance of about 30 cm from the person's eyes in natural daylight conditions.

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include webs or layers or fibrous materials, films and foils such as plastic films or metallic foils that may be used alone or laminated to one or more web, layer, film and/or foil.

The term “CIELAB color scale or space” refers herein to a color space that encompasses RGB and CMYK, and describes generally the visible spectrum that the human eye can see. In the CIELAB space, a color may be defined by three parameters L*, a* and b* where L* represents relative luminance, a* represents relative redness-greenness and b* represents relative yellowness-blueness.

The term “color” as referred to herein include any primary color, i.e., white, black, red, blue, violet, orange, yellow, green, and indigo as well as any declination thereof or mixture thereof within the CIELAB color space or scale.

The term “background color” refers herein to the color of the substrate.

The term “white” refers herein to those colors having an L* value of at least 90, an a* value equal to 0±3, and a b* value equal to 0±3 (in terms of the Commission Internationale d'Eclairage, 1976 L*, a*, b* color scale, i.e. CIELAB). The term “repeating pattern” is used herein to refer to a pattern that may include at least about 10 macro-units having substantially the same overall shape.

The term “substrate with an actual three-dimensional pattern or texture” is used herein to refer to a substrate having a pattern exhibiting noticeable variations in its topography as opposed to a substrate which is substantially flat. A person may be able to see this actual three-dimensional pattern. A person may also be able to notice and/or feel the three-dimensional pattern topography by passing a finger across the pattern on the substrate.

The term “substrate with perceived three-dimensional pattern or texture” is used herein to refer to a substrate having a pattern which does not exhibit a noticeable variation in topography but is nevertheless perceived by a viewer as being three-dimensional. Although a person may be able to see this perceived three-dimensional pattern, a person may not be able to notice and/or feel the three-dimensional pattern topography by passing a finger across the pattern on the substrate.

As used herein the term “stretchable” refers to materials which can stretch to at least an elongated length of 105% on the upcurve of the hysteresis test at a load of about 400 gm/cm. The term “non-stretchable” refers to materials which cannot stretch to at least 5% on the upcurve of the hysteresis test at a load of about 400 gm/cm.

The terms “elastic” and “elastomeric” as used herein refer to any material that upon application of a biasing force, can stretch to an elongated length of at least about 110%, preferably to 125% of its relaxed, original length (i.e. can stretch to 10 percent, preferably 25% more than its original length), without rupture or breakage, and upon release of the applied force, recovers at least about 40% of its elongation, preferably recovers at least 60% of its elongation, most preferably recovers at least about 80% of its elongation. For example, a material that has an initial length of 100 mm can extend at least to 110 mm, and upon removal of the force would retract to a length of 106 mm (40% recovery). The term “inelastic” refers herein to any material that does not fall within the definition of “elastic” above.

The term “extensible” as used herein refers to any material that upon application of a biasing force, can stretch to an elongated length of at least about 110%, preferably 125% of its relaxed, original length (i.e. can stretch to 10 percent, preferably 25% more than its original length), without rupture or breakage, and upon release of the applied force, shows little recovery, less than about 40%, preferably less than about 20% and more preferably less than about 10% of its elongation.

The term “flexible” means herein that the material may tend to conform or deform in the presence of externally applied forces. As measured under the Stiffness of Fabric test, described herein, a flexible sheet material may have a peak load of less than about 1000 g_{f}.

The term “rigid” means herein that the material may tend to resist deformation in the presence of externally applied forces. As measured under the Stiffness of Fabric test, described herein, a rigid material may have a peak load of greater than 1000 g_{f}.

While not intending to limit the utility of the invention herein, it is believed that a brief description of its use will help elucidate the invention. The literature is replete with substrates that are modified to include an actual three-dimensional pattern. These actual three-dimensional patterns are believed among other things to increase consumers' appeal to the substrate. However, modifying a substrate in order to provide an actual three-dimensional pattern thereto also comes with many disadvantages such as cost (in material and equipment), deterioration of the substrate properties (e.g. strength) and a limited capacity to modify the pattern shape or design in response to product trends. The literature is also replete with substrates that include graphics such as designs, characters, icons, and the like which may also make the substrate more aesthetically appealing to consumers. However, although the graphic itself may appear to be three-dimensional, a person such as a consumer looking at the graphic printed on a substrate may not perceive and/or believe that the substrate itself is three-dimensional. It is found that the consumers' appeal for an article that includes a substrate may be improved by providing a substrate with a perceived three-dimensional repeating pattern that may be printed on the substrate as opposed to an actual three-dimensional repeating pattern that is physically formed on the substrate. Among other benefits, it is believed that by printing a perceived three-dimensional pattern on a substrate, the manufacturing cost of the substrate may be reduced, the mechanical properties of the substrate may not be altered and a manufacturer may have more options and flexibility when the manufacturer wishes to change the pattern design, shape and/or color.

Aspects of the present disclosure involve printing a repeating pattern on a substrate such as a nonwoven web or fabric, plastic film, and laminate thereof in order to provide this substrate with a perceived three-dimensional pattern, which may cause a visible surface of the substrate to exhibit a three-dimensional appearance. In some embodiments, the three-dimensional appearance of the substrate surface resembles protrusions and indentions indicative of threads in woven cloths. The patterns are created by printing a surface of a substrate, as opposed to altering or deforming the substrate such as by embossing or hydro-molding. As discussed in more detail below, embodiments of the patterns include a plurality of repeating shapes or macro-units disposed on the substrate surface. Each macro-unit has three or more color zones. In some embodiments, all the color zones are defined by printed colors. In other embodiments, one color zone may be defined by the substrate color or background color with the remainder of the color zones being printed on the substrate. The color zones have different levels of contrast, wherein the color zones transition from a darkest to lightest. The color zones may also have different shapes and sizes, defining different shapes and sizes of the macro-units. The macro-units, when arranged to form a repeating pattern, define brighter and darker areas on the substrate surface. The brighter and darker areas give the appearance that light is brightly shining on the peaks of raised areas protruding from the substrate surface. In addition, the raised areas appear to be casting shadows on other areas such as valleys of the substrate. As such, the patterns give the substrate the appearance of having three-dimensional surface characteristics that provide the substrate with a perceived three-dimensional cloth-like appearance.

Various characteristics and parameters of the patterns can be varied to provide a perceived three-dimensional appearance to the substrate surface as well as the individual macro-units. As discussed in more detail below, the size of the individual macro-units, the number of zones in the individual macro-units, and the contrast levels between the color zones may be varied based on the size of the substrate and the distance from which the substrate is to be viewed to provide a desired three-dimensional appearance. In one example, an individual macro-unit may require additional color zones in order for the macro-unit to appear three-dimensional as the size of the individual macro-unit is increased for a given viewing distance. In another example, fewer color zones may be required in order for the macro-unit to appear three-dimensional as the viewing distance is increased for a given macro-unit size.

As previously mentioned, patterns according to the present disclosure may have color zones that are printed on substrates. As such, the contrast levels between the color zones of the macro-units that form the perceived repeating pattern can be achieved in various ways. In one example, the macro-units are printed with more than one ink having different levels of darkness. More particularly, a first ink may be used to print a first color zone, and a second ink that is brighter (i.e. having a higher L* value) than the first ink may be used to print a second color zone. In another example, the macro-units are printed with a single ink wherein a thicker coat or more coats of the ink are used to print a first zone than a second zone. As such, the first zone appears darker than the second zone. In yet another example, a first zone may be darker than a second zone by printing both zones with the same ink but printing the first zone with a higher dot or micro-unit density than the second zone. In addition to the contrast levels, the size and shape of the macro-units and color zones may vary to achieve a desired appearance. For example, in some embodiments, the color zones are printed such that the resulting macro-units have an asymmetrical shape. It is believed that macro-units having an asymmetrical shape may cause the substrate to appear and be perceived as three-dimensional by having a plurality of raised areas arranged in a pattern. In some embodiments, the macro-units and color zones are sized and shaped in order to simulate the light effect on an actual three-dimensional pattern when light impacts the raised areas formed on the substrate surface at a relatively small acute angle relative the substrate surface. In addition, the raised areas may appear to be casting relatively long shadows on other areas of the substrate surface. Although the many pattern embodiments are discussed herein with the perspective that the substrate background color has a relatively high L* value in comparison to the printed colors which are relatively dark, it is to be appreciated that in some embodiments the substrate background color may be relatively dark (i.e. it may have a low L* value) and the printed colors may be relatively light (i.e. the printed colors may have a greater L* value than the background color).

Printing may be characterized as an industrial process in which an image is reproduced on a substrate, such as paper, polyolefin film, or nonwoven fabric. There are various classes of printing processes, which may include stencil and screen printing, relief printing, planographic printing, intaglio printing, and electronic printing. Stencil and screen printing may be used for printing T-shirts, signage, banners, billboards, and the like. Examples of relief printing may include letterpress and flexography. Examples of planographic printing may include offset lithography, screenless lithography, collotype, and waterless printing. In addition, examples of intaglio printing may include gravure, steel-die, and copper-plate engraving. Examples of electronic printing may include electrostatic, magnetographic, ion or electron deposition, and ink-jet printing. It is it to be appreciated that various types of printing processes may be used to create the patterns disclosed herein. For example, in some embodiments, it may be preferable to use flexography. In particular, flexography may utilize printing plates made of rubber or plastic with a slightly raised image thereon. The inked plates are rotated on a cylinder which transfers the image to the substrate. Flexography may be a relatively high-speed print process that uses fast-drying inks. In addition, flexography can be used to print continuous patterns on many types of absorbent and non-absorbent materials. Other embodiments may utilize gravure printing. More particularly, gravure printing utilizes an image etched on the surface of a metal plate. The etched area is filled with ink and the plate is rotated on a cylinder that transfers the image to the substrate. Still other embodiments may utilize ink-jet printing. Ink-jet is a non-impact dot-matrix printing technology in which droplets of ink are jetted from a small aperture directly to a specified position on a media to create an image. Two examples of inkjet technologies include thermal bubble or bubble jet and piezoelectric. Thermal bubble uses heat to apply to the ink, while piezoelectric uses a crystal and an electric charge to apply the ink.

In addition to the aforementioned various types of printing processes, it is to be appreciated that various types of inks or ink systems may be applied to various types of substrates to create the disclosed patterns, such as solvent-based, water-based, and UV-cured inks. The primary difference among the ink systems is the method used for drying or curing the ink. For example, solvent-based and water-based inks are dried by evaporation, while UV-cured inks are cured by chemical reactions. Inks may also include components, such as solvents, colorants, resins, additives, and (for ultraviolet inks only) UV-curing compounds, that are responsible for various functions.

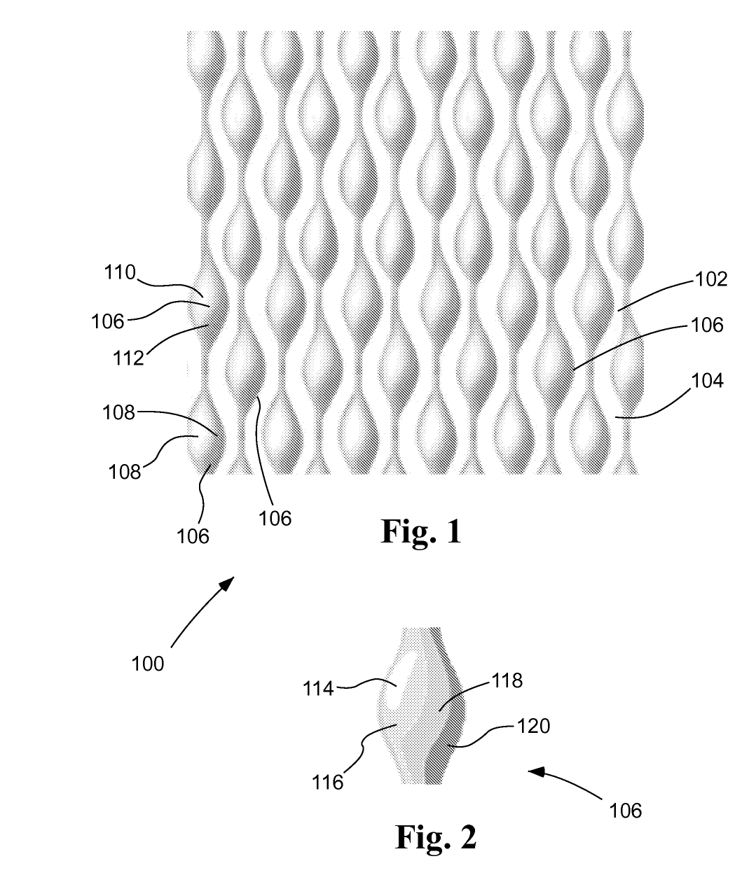

FIG. 1 shows one example of a pattern 100 that may be disposed on a surface 102 of a substrate 104 to provide a three-dimensional appearance to the substrate surface. As shown in FIG. 1, the pattern 100 includes a plurality of repeating shapes or macro-units 106 disposed on the substrate surface 102. As discussed in more detail below, each macro-unit 106 may have three or more color zones 108 having different levels of contrast, wherein the color zones 108 transition from a darkest to lightest. As previously mentioned, one color zone may be defined by the substrate background color with the remainder of the color zones being printed. Alternatively, all the color zones may be defined by printed colors. As shown in FIG. 1, the color zones 108 define brighter areas 110 and darker areas 112 on the substrate surface 102. The brighter areas 110 give the appearance that light is intensely reflected (i.e. perceived as brightly shining) from the raised areas protruding from the substrate surface. In addition, the darker areas 112 give the appearance that raised areas are casting shadows on other areas (i.e. valleys) of the substrate. As such, the pattern gives the substrate the appearance of having three-dimensional surface characteristics that may be perceived by a person.

As discussed in more detail below, the patterns disclosed herein, such as the pattern 100 shown in FIG. 1, may be printed on substrates that may be incorporated into a variety of items in order to provide a desired perceived three-dimensional or cloth-like appearance. For example, patterns may be disposed on nonwoven fabrics, films, foils and/or laminates thereof used in many articles. Non-limiting examples of such articles include absorbent articles for inanimate surfaces, absorbent articles for animate surfaces and packages. Without intending to limit the scope of the invention, patterns may be disposed on nonwoven fabrics, films and/or laminates thereof that are used to manufacture absorbent articles for animate surfaces such as diapers. In this embodiment the pattern may be disposed on that the substrate used as the outer and/or inner layers of the absorbent articles in order to provide this layer(s) with a perceived three-dimensional a cloth-like appearance. In other examples, medical products, such as surgical gowns, drapes, face masks, head coverings, shoe coverings, wound dressings, bandages and sterilization wraps, may utilize substrates with the disclosed patterns such that the medical products also exhibit a perceived three-dimensional cloth-like appearance. In yet other examples, packaging used to hold various types of products may be constructed with substrates having patterns disposed thereon that provide a perceived three-dimensional pattern or texture to the package. In some instances, it may be preferable to print such patterns on substrates that are flexible and/or exhibit flexibility, which may allow the substrate to conform to a particular shape, such as a person's body or a package. Some such flexible substrates sheet material may have a peak load of less than about 1000 g_{f}, while others may have a peak load of less than about 250 g_{f}, and still others may have a peak load of less than about 10 g_{f}, as measured under the Stiffness of Fabric test, described herein. It is to be appreciated that various types of nonwoven fabrics, films, and/or laminates constructed from various materials and having various basis weights may be used. Examples of nonwovens may include polypropylene (i.e. PP), polyethylene (i.e. PE), or copolymers of the same, with basis weights from 5 grams per square meter up to 60 grams per square meter. In addition, examples of film substrates may include PP, PE, or copolymers of the same, breathable and non-breathable films, with basis weights of from 5 grams per square meter up to 50 grams per square meter.

It is to be appreciated that embodiments of patterns according to the present disclosure have various properties that may be varied to provide a perceived three-dimensional or cloth-like appearance to a substrate surface upon which the patterns are printed. Such properties may include at least one of: the number of color zones in each macro-unit; the contrast levels between adjacent color zones; the macro-unit sizes; the maximum distances between adjacent macro-units; and any combinations thereof. As previously mentioned, FIG. 1 shows one embodiment of a perceived three-dimensional repeating pattern 100 that may be used to provide the perceived three-dimensional and/or cloth-like appearance to a substrate surface. The repeating pattern 100 is defined by an arrangement of macro-units 106, each macro-unit 106 having at least three color zones 108. FIG. 2 shows one embodiment of a macro-unit 106 including a first color zone 114, a second color zone 116, a third color zone 118, and fourth color zone 120. In the embodiment shown in FIG. 2, the first color zone 114 corresponds with the substrate background color, while the second, third, and fourth color zones 116, 118, 120 are printed on the substrate. However, as previously mentioned, all the color zones may be printed on the substrate. As discussed in more detail below, the color zones have different levels of contrast. More particularly, the fourth color zone 120 is darker than the third color zone 118; the third color zone 118 is darker than the second color zone 116; and the second color zone 116 is darker than the first color zone 114. The different levels of contrast between the zones gives the macro-unit the appearance that light is shining more brightly on the relatively brighter first color zone 114 and that shadows are being cast on the relatively darker fourth color zone 120. The second and third color zones 116, 118 provide a relatively smooth transition between the first color zone 114 and the fourth color zone 120. The appearance of the bright areas and dark shaded areas gives each macro-unit the perceived appearance of three-dimensionality. In turn, a plurality of the macro-units arranged in a pattern on a substrate give the substrate surface the perceived appearance of three-dimensionality.

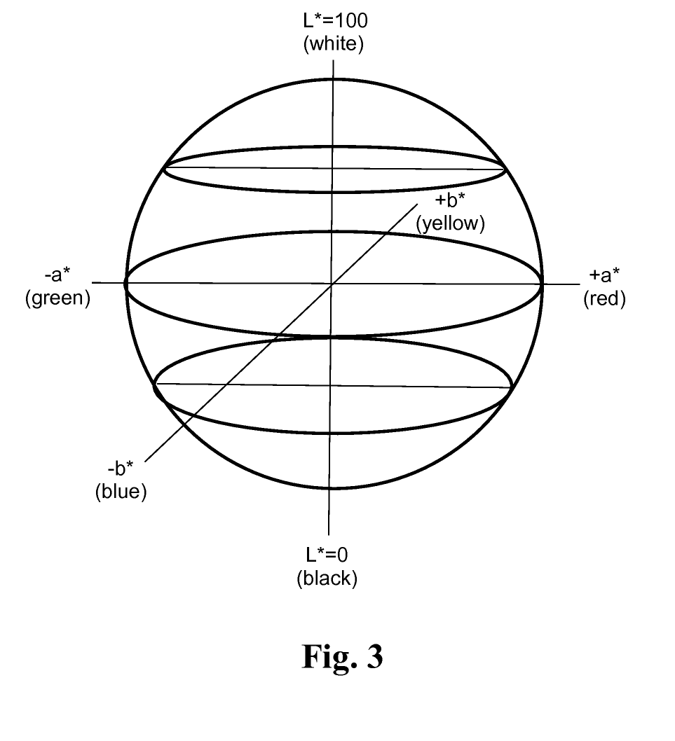

As previously mentioned, the contrast levels between the color zones may vary. The following provides a discussion of how the levels of contrast between the color zones can be quantified. In particular, the levels of contrast between the zones of the macro-units are defined in terms of L* values based on the CIELAB color scale. CIELAB is a conventional color model used to describe colors visible to the human eye. FIG. 3 is an illustration of three axes (respectively for the L*, a*, and b* value of a given color) used with the CIELAB color scale. When a color is defined according to the CIELAB color scale, L* represents lightness (0=black, 100=white), a* and b* independently each represent a two color axis, a* representing a red/green axis (+a=red, −a=green), while b* represents a yellow/blue axis (+b=yellow, −b=blue). The maximum for L* is 100, which represents a perfect reflecting diffuser, and the minimum for L* is zero, which represents black. The a* and b* axes have no specific numerical limits. The CIELAB color scale is an approximate uniform color scale, wherein the differences between points plotted in the color space correspond to visual differences between the colors plotted. Based on the L*, a*, and b* values for a first color (i.e. L1, a1, b1) and a second color (i.e. L2, a2, b2), the difference between the colors (i.e. ΔE) can be calculated using the following formula:

ΔE=(ΔL*²+Δa*²+Δb*²)^{1/2}

wherein,
ΔL*=L₁−L₂;
Δa*=a₁−a₂; and
Δb*=b₁−b₂.

It is to be appreciated that the contrast levels between the color zones of the macro-units discussed herein may be defined by ΔL* without regard to the values of Δa* and Δb*. As such, pattern embodiments according the present disclosure may have different Δa* and Δb* values. In some embodiments, the colors of the printed zones and the substrate may have a* and b* values that are approximately the same, wherein the Δa* and Δb* are relatively low values (e.g. Δa*=±5, and Δb*=±5). In such an embodiment, the difference between the colors of the individual zones as well as the substrate can also be approximated by the difference between the L* values (i.e. ΔL*) of the colors. In other embodiments, a*=b*=0, with the L* axis representing the achromatic scale of grays from black to white. The L* values for the color zones may be determined in various ways. For example, the L* values of the color zones may be determined by using ink with relatively known L* values. Alternatively, the L* values on a macro-unit can be determined from the electronic file that is generated when a pattern is created. In such a case, the L* values may be obtained with a computer equipped with a software that can provide the L* value of a selected area. A non-limiting example of such a software may be Adobe Photoshop®. In another embodiment, the L* values of various color zones on a macro-unit can be measured directly from the printed substrate. A procedure for measuring the L* values of a color zone is provided below.

It is to be appreciated that there may be limits on the ΔL* values between the color zones in order to give a macro-unit a desired perceived three-dimensional appearance. For example, if the ΔL* values between the darkest color zone and the brightest color zone of a macro-unit are too small, it may be relatively difficult for a human eye to discern the different contrast levels between the lightest and darkest color zones as well as any color zones in between. As such, the macro-unit may appear to be of one color without any contrast transition, and thus may not be perceived as being three-dimensional by a person. It will be appreciated by one of skill in the art that when a substrate defines a background color with a relatively high L* value (i.e. relatively light) and if the ΔL* value between the background color and the darkest color zone of the macro-unit is too small, the macro-unit may not be discernable by a viewer. It will also be appreciated that when the substrate defines a background color with a relatively low L* value (i.e. relatively dark) and if the ΔL* value between the background color and the brightest color zone of the macro-unit is too small, the resulting macro-unit may not be discernable by a viewer. In another example, when transitioning from a zone having the highest L* value (i.e. the lightest zone) to an adjacent color zone that is relatively darker, the ΔL* values between the two color zones may be so large that the contrast levels between the two color zones may not have a smooth contrast transition. As a result, the macro-unit may not be perceived as being three-dimensional.

The following guidelines provide ΔL* limits between zones in pattern embodiments wherein each macro-unit has three color zones. Such pattern embodiments may have a first color zone with a L* value of L1, a second color zone with a L* of L2, and a third color zone with a L* value of L3, and wherein L1>L2>L3. In such pattern embodiments, the difference between L1 and L3 must be greater than or equal to 3, while the difference between L1 and L2 must be greater than or equal to 2 and less than or equal to 10. In other words, for pattern embodiment having macro-units with no more than three color zones defining L* values of L1, L2, and L3 (wherein L1>L2>L3), the following limits on L* may be applied:

3≦(L1−L3); and

2≦(L1−L2)≦10

The following guidelines provide ΔL* limits between zones in pattern embodiments wherein each macro-unit has more than three adjacent color zones progressively ranging from the highest L* value (the brightest) to the lowest L* (the darkest). In such embodiments, the ΔL* value between the brightest zone and the next darkest zone may be between 2 and 10 (inclusive). The ΔL* between subsequent adjacent zones may be at least 2 (inclusive). In other words, for pattern embodiments having macro-units with N color zones (N being an integer), wherein N>3 and the zones define L* values of L₁, L₂, L₃, . . . , and L_{N} (wherein L₁>L₂>L₃> . . . >L_{N}), the following limits on L* may be applied:

$$2 \leq ( {L}_{1} - {L}_{2} ) \leq 10 ;$$

$$2 \leq ( {L}_{2} - {L}_{3} ) ;$$

$$and$$

$$\begin{matrix}⋮ \\ 2 \leq ( {L}_{N - 1} - {L}_{N} )\end{matrix}$$

In one example, a macro-unit has four color zones (e.g. a first color zone with a L* value of L1, a second color zone with a L* of L2, a third color zone with a L* of L3, and a fourth color zone with a L* value of L4, and wherein L1>L2>L3>L4). In such a pattern embodiment, the difference between L1 and L2 may be greater than or equal to 2 and less than or equal to 10, while the difference between L2 and L3 may be greater than or equal to 2. In addition, the difference between L3 and L4 may be greater than or equal to 2.

It is to be appreciated that various substrate characteristics may also have an affect on the L* values of printed color zones. For example, when a pattern is printing on the surface of a substrate, the substrate thickness and/or substrate color may “dilute” the L* values of inks used to create the printed color zones. In such an example, inks with relatively higher L* values may be used to create patterns having color zones that fall within the previously disclosed limits on L* values between color zones.

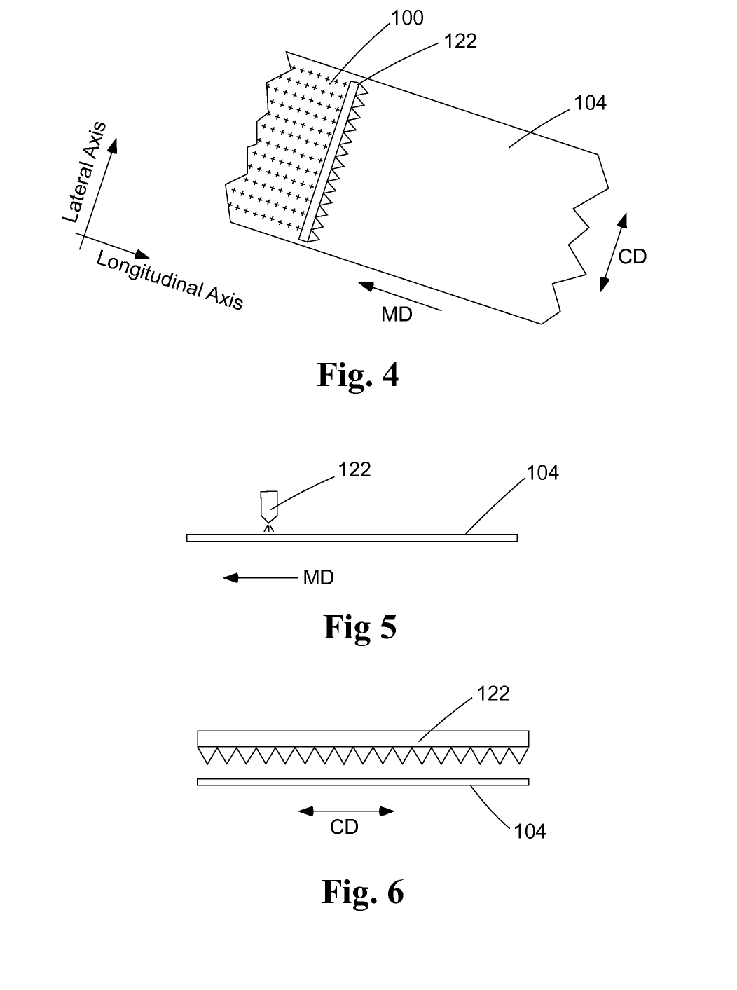

As previously mentioned, the macro-units making up the patterns have at least three color zones. It is to be appreciated that macro-units may have more than three color zones as discussed below. In some embodiments, all of the color zones are printed on a substrate. In other embodiments, one of the color zones is defined by the substrate background color with the remainder of the zones being defined by colors that are printed on the substrate. The L* values of the color zones range from a relatively high value (brightest) to a relatively low value (darkest). As previously mentioned, the color zones may have different shapes and sizes, defining different shapes and sizes of the macro-units. FIGS. 4-6 shows one example of how a pattern 100 may be printed on a substrate. The pattern in FIG. 4 is schematically represented by a series of “+” shapes. To provide a frame of reference for the present discussion, the substrate 104 is shown in FIG. 4 with a longitudinal axis and a lateral axis. The longitudinal axis also corresponds with what may be referred to as the machine direction (i.e. MD) of the substrate, and the lateral axis corresponds with what may be referred to as the cross direction (i.e. CD) of the substrate. As shown in FIGS. 4-6, a pattern 100 may be printed on a substrate 104 by moving the substrate in the longitudinal direction shown relative to a printing device 122, such as those referenced above, while the printing device 122 prints the desired printed colored zones of each macro-unit. It is to be appreciated that the printing device may also move relative to the substrate while printing. For example, the printing device may move back and forth in lateral directions relative to the substrate while printing the desired printed colored zones of each macro-unit.

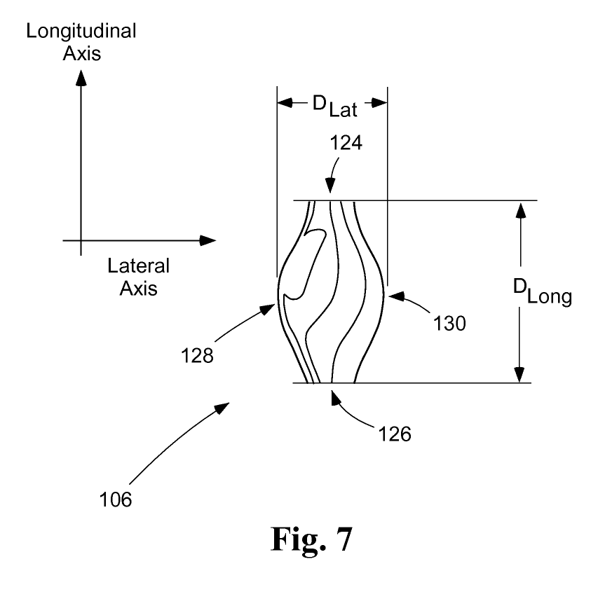

It is to be appreciated that a multitude of macro-unit shapes can be used in a multitude of pattern embodiments, and as such, a multitude of macro-unit sizes or areas may be used. The present disclosure characterizes the macro-unit size by a macro-unit's primary dimension (referred to as U_{pd}), which is defined by the following description. FIG. 7 is detailed enlarged view of an example single macro-unit 106 from a repeating pattern 100. It is to be appreciated that the actual primary dimension of the macro-unit shown in FIG. 7 may vary. As shown in FIG. 7, the macro-unit 106 includes a first longitudinal print point 124 and a second longitudinal print point 126, and defining a distance (i.e. D_{long}) therebetween. No portion of the macro-unit 106 is printed in longitudinal directions outside the distance (i.e. D_{long}). The macro-unit 106 also includes a first lateral print point 128 and a second lateral print point 130, and defining a distance (i.e. Dₗₐₜ) therebetween. No portion of the macro-unit 106 is printed in lateral directions outside the distance (i.e. Dₗₐₜ). In other words, distance D_{long} represents the maximum length of the printed zones of the macro-unit in the longitudinal direction, and the distance Dₗₐₜ represents the maximum length of the printed zones of the macro-unit in the lateral direction. As such, the actual primary dimension (i.e. U_{pd}) may be defined as the minimum of D_{long} and Dₗₐₜ. For example, if a macro-unit has a D_{long} of 4 mm and a Dₗₐₜ of 1.5 mm, the primary dimension of the macro-unit is said to be 1.5 mm. When D_{long} and Dₗₐₜ are equal, the primary dimension may be defined as the distance represented by either D_{long} of Dₗₐₜ. For example, if a macro-unit has a D_{long} of 1.5 mm and a Dₗₐₜ of 1.5 mm, the primary dimension is said to be 1.5 mm. In one embodiment, the actual primary dimension U_{pd} of a macro-unit is at least 1.5 mm.

As previously mentioned, there is a relationship between the actual sizes of the macro-units, the distance from which the macro-units are viewed by a person, and the number of color zones in each macro-unit in order for the macro-units to provide a relatively smooth transition between light and dark color zones so as to exhibit a perceived three-dimensional appearance. Without intending to be bound by any theory, it is believed that when a person looks at a repeating pattern from a relatively close viewing distance (i.e. less than 30 cm), this person's eyes can more easily detect specific details of the macro-units (e.g. the individual color zones). It is also believed that from the same relatively close viewing distance, the person's eyes may not be as easily able to notice the specific details of a repeating pattern that includes relatively small macro-units as compared to relatively large macro-units. As such, it is believed that a relatively small macro-unit forming a repeating pattern may not require as many color zones as a relatively large macro-unit may require when viewed from a relatively close distance in order to provide a smooth transition between light and dark zones. In addition, it is believed that when a person looks at a repeating pattern from a relatively far viewing distance (i.e. more than 30 cm), the person's eyes may not as easily notice specific details of the macro-units (e.g. the individual color zones). In addition, from a relatively far viewing distance, a person's eyes may not as easily notice specific details of relatively large macro-units as he or she would have otherwise noticed from relatively close viewing distances. As such, it is believed that a relatively large macro-unit may not require as many color zones when viewed from a relatively far distance in order to provide a smooth transition between bright and dark zones.

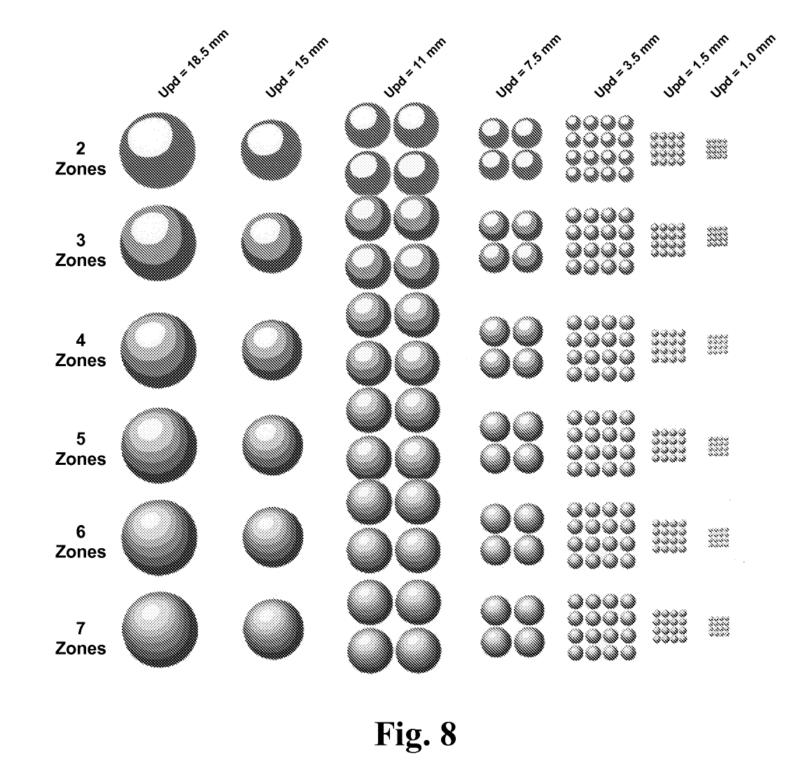

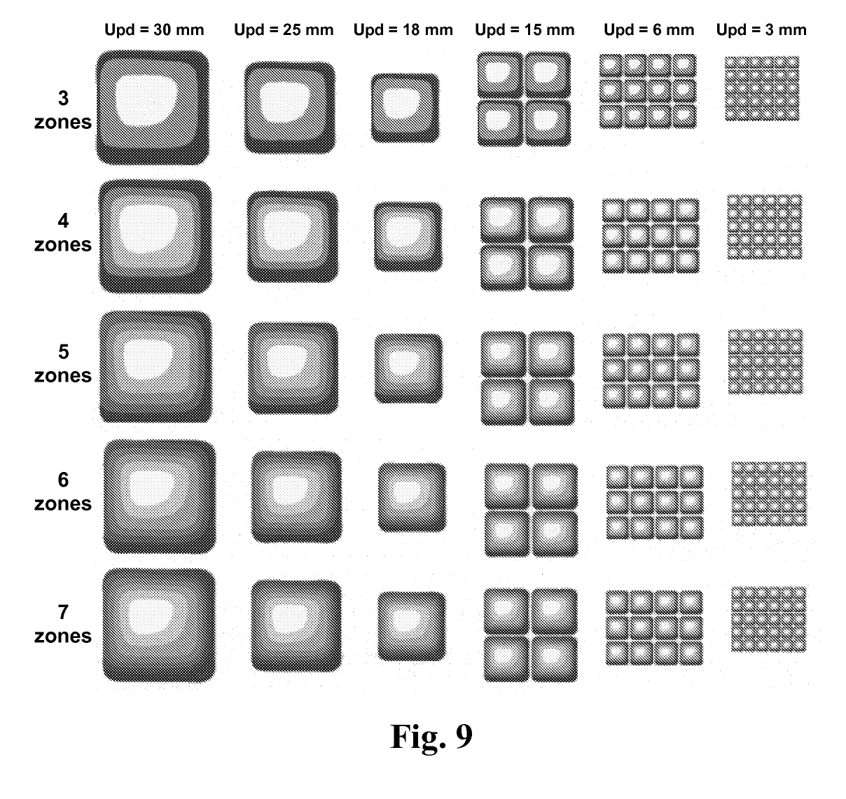

The foregoing discussion may be illustrated by viewing the plurality of macro-units shown FIGS. 8 and 9 from various distances. For reference purposes, the macro-units are arranged by rows and columns. The rows correspond to the number of zones in each macro-unit ranging from 3 to 7, and the columns correspond to variations of the actual primary dimension of the macro-units ranging from relatively large (left columns) to relatively small (right columns). It is believed that depending on the “distance of interaction” between a person and a device or object, some of the parameters defining the macro-units of a repeating pattern may be adjusted such that the macro-units are perceived as three-dimensional from this “distance of interaction.” It will be appreciated that people “interact” with and consequently look at various devices or objects from various distances. By way of example, the user of an absorbent article for animate surfaces may look at (and interact with) this article from a distance of 20 cm to 1 m (from removing the article from its package to actual use). A person walking in the aisles of a store and looking at products placed on the store's shelves may look at these products from a greater distance. It is believed that FIGS. 8 and 9 may help the reader understand the relationship between these parameters (for example the number of color zones, the actual primary dimension of the macro-unit as well as the perceived primary dimension of the macro-unit) and perceived three-dimensional effect. It should be noted that the macro-units 106 shown in FIGS. 8 and 9 are for illustration purpose only. Based on the foregoing discussion, it may be desirable to determine how many zones may be included in the macro-units based on an estimated distance of interaction. The estimated distance of interaction may be based on a number of factors, such as how and where a particular substrate may be applied. For example, when applying presently disclosed patterns to the outer cover of a diaper that may be viewed by a caregiver from a relatively close distance, it may be desirable to estimate a distance of interaction that is relatively small. In other applications, such as when applying the printed pattern to a package such that it is visible on the outer surface of a package displayed on a store shelf, it may be desirable to estimated a distance of interaction that is relatively large.

The following guidelines can be used to determine the number of color zones for each macro-unit based on the macro-unit actual size and distance of interaction. As previously mentioned, the macro-unit size can be characterized by the macro-unit's actual primary dimension U_{pd}. In particular, Table 1 below provides a guideline as to the number of zones (i.e. N_{zone}) required per macro-unit based on the actual primary dimension (i.e. U_{pd}), assuming a distance of interaction (i.e. L_{dist}) of 30 cm:

TABLE 1
U_{pd} vs. N_{zone}, wherein I_{dist} = 30 cm

| Primary Dimension | |
|---|---|
| (mm) | No. of Zones N_{zone} |
| U_{pd} = 1.5 | 3 |
| 1.5 < U_{pd} ≦ 2.5 | 4 |
| 2.5 < U_{pd} ≦ 5 | 5 |
| 5.0 < U_{pd} ≦ 22 | 6 |
| 22 < U_{pd} ≦ 28 | 7 |

Using Table 1 above, a macro-unit having an actual primary dimension of 1.5 mm when viewed from a distance of 30 cm, may require at least 3 color zones. In another example, a macro-unit having an actual primary dimension of 5 mm when viewed from a distance of 30 cm, may require at least 5 color zones. Although the maximum U_{pd} value provided in Table 1 is 28 mm, it is to be appreciated that larger U_{pd} values may be achieved, and as such, may require additional zones.

As previously mentioned, fewer numbers of zones are required for a particular macro-unit primary dimension as the distance of interaction increases. The N_{zone} values provided above in Table 1 are based on an distance of interaction (I_{dist}) of 30 cm. Other N_{zone} values may be calculated for various distances of interaction assuming that there is an inverted relationship between the number of required zones and the distance of interaction. In other words, the N_{zone} values presented in Table 1 can be multiplied by a ratio of 30 cm over a desired distance of interaction to adjust the number of color zones for the desired distance of interaction so long as the N_{zone} value is greater than or equal to 3, as represented by the following equation:

N_{zone}=(N_{zone} of Table 1)*(30 cm)/(I_{dist}), and N_{zone}≧3

In one example wherein the distance of interaction for a particular pattern is 60 cm, the number of required zones (N_{zone}) for a macro-unit having an actual primary dimension (U_{pd}) of 11 mm can be calculated as follows:

N_{zone}=(6 zones)*(30 cm)/(60 cm)=3 zones.

As such, a macro-unit with an actual primary dimension of 11 mm when viewed from a distance of 60 cm may only require 3 zones to achieve a perceived three-dimensional effect. In an another example wherein the distance of interaction for a particular pattern is 60 cm, the number of required zones (N_{zone}) for a macro-unit having a primary dimension of 2.5 mm can be calculated as follows:

N_{zone}=(4 zones)*(30 cm)/(60 cm)=2 zones.

However, as discussed above, it may be preferably for N_{zone} to be greater than or equal to 3. As such, a pattern having a macro-unit actual primary dimension of 2.5 mm when viewed from a distance of 60 cm may also require at least 3 color zones.

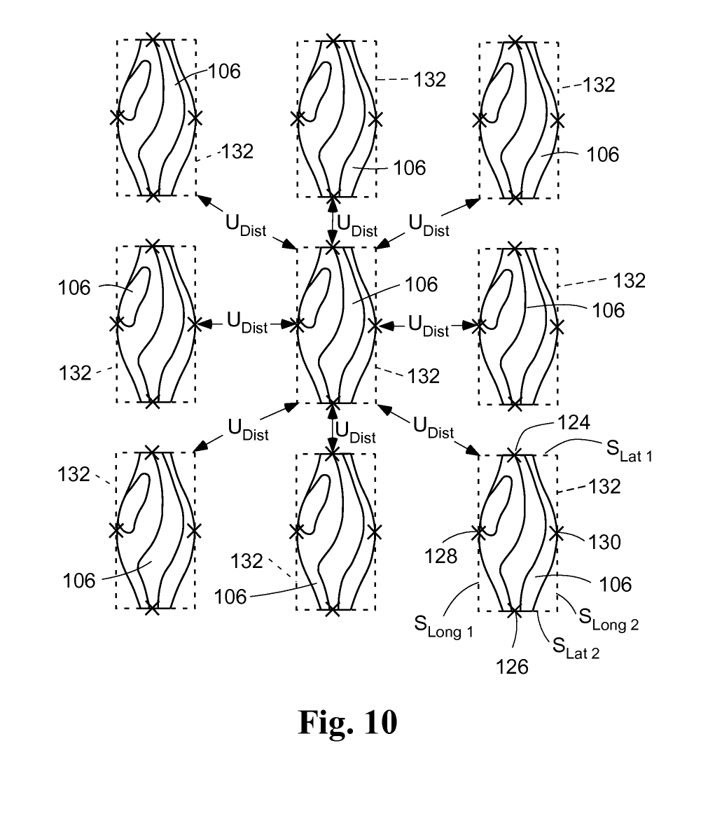

As previously mentioned, the distances between adjacent macro-units of a pattern may have an effect on whether a substrate surface exhibits a perceived three-dimensional and/or cloth-like appearance. For example, if the distances between adjacent macro-units are too large, the human eye may be more apt to focus on individual macro-units as opposed to the pattern as a whole, and as such, the macro-units and/or the substrate surface may not exhibit a perceived three-dimensional appearance. The distances (U_{dist}) between adjacent macro-units of a pattern may be estimated by measuring the shortest space between print point rectangles or squares drawn around adjacent macro-units. As shown in FIG. 10, each macro-unit 106 is surrounded by a print point rectangle 132. Each print point rectangle 132 is defined by two longitudinally extending sides (S_{long1}, S_{long2}) and two laterally extending sides (Sₗₐₜ₁, Sₗₐₜ₂). The longitudinally extending sides (S_{long1}, S_{long2}) are also tangentially related to the first lateral print point 128 and the second lateral print point 130, respectively, of the macro-unit. Similarly, the laterally extending sides (S_{long1}, S_{long2}) are also tangentially related to the first longitudinal print point and the second longitudinal print point, respectively, of the macro-unit.

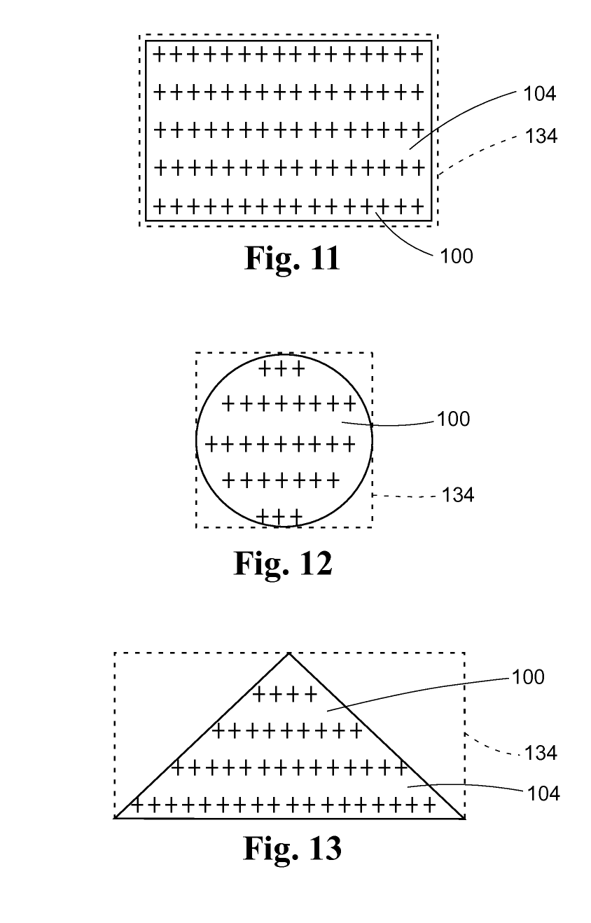

The following procedure and examples illustrated in FIGS. 11-13 are set forth to aid in determining the maximum distances between adjacent macro-units 106 in a repeating pattern. To determine the maximum distance between adjacent macro-units 106 in a pattern 100, the substrate 104 with the pattern disposed thereon is placed within a theoretical rectangle or square 134. This theoretical rectangle or square 134 should define the smallest possible rectangle or square that contains the printed perimeter of the substrate 104. The actual lengths of the sides of the rectangle are then measured to determine the length of the longest side of the rectangle. The maximum distance between adjacent macro-units is then calculated by multiplying an aspect ratio by the actual length of the longer side of this theoretical rectangle. By way of example, if the substrate printed perimeter defines a shape that fits within a square, the actual length of any side of the square may be used. For the purposes of this discussion, the aspect ratio can be 0.1. The examples provided below illustrate how the maximum distance between adjacent macro-units may be calculated using the aforementioned procedure with substrates having various shapes or printed perimeter having a shape other than the shape of the substrate.

FIG. 11 illustrates an embodiment of a substrate 104 having an outer perimeter that defines a rectangular shape having four sides. A repeating pattern 100 of macro-units (schematically represented by an arrangement of “+” shapes) is printed substantially across the whole substrate 104. Because the outer perimeter of the substrate defines a rectangular shape, the smallest possible theoretical rectangle or square 134 that can contain the whole substrate matches the size and shape of the outer perimeter of the substrate. Using the aforementioned procedure, the actual lengths of the sides of the theoretical rectangle 134 are measured to determine the actual length of the longest sides. The actual length of the longest sides is then multiplied by 0.1 to calculate the maximum distance between macro-units. In one example, the rectangle includes two sides having an actual length of 10 cm and two sides having an actual length of 15 cm. As such, the maximum distance between immediately adjacent and consecutive macro-units is calculated by multiplying 15 cm by 0.1, which equates to 1.5 cm.

FIG. 12 illustrates another embodiment of a substrate 104 printed with a repeating pattern 100 (schematically represented by an arrangement of “+” shapes) having an outer perimeter that defines a circular shape. Because the outer perimeter of the substrate defines a circular shape, one of ordinary skill will understand that a square can contain the substrate having side actual lengths that match the diametrical actual length of the circle. Using the aforementioned procedure, the actual length of the sides of the theoretical square 134 is measured. The actual length of the sides can then be multiplied by 0.1 to calculate the maximum distance between macro-units. In one example, the square includes four sides having an actual length of 5 cm. As such, the maximum allowable distance between macro-units is calculated by multiplying 5 cm by 0.1, which equates to 0.5 cm.

FIG. 13 illustrates yet another embodiment of a substrate 104 having an outer perimeter that defines a triangular shape having three sides. The substrate 104 is then placed within the smallest possible theoretical rectangle 134. Using the aforementioned procedure, the actual lengths of the sides of the rectangle 134 are measured to determine the actual length of the longest sides. Again, the actual length of the longest sides is then multiplied by 0.1 to calculate the maximum distance between macro-units. In one example, the rectangle includes two sides having a length of 4 cm and two sides having a length of 8 cm. As such, the maximum allowable between macro-units is calculated by multiplying 8 cm by 0.1, which equates to 0.8 cm.

Although the aforementioned discussion relates to determining a maximum distance between immediately adjacent and consecutive macro-units of a pattern, it is to be appreciated that in some pattern embodiments, adjacent macro-units may be in contact with each other. In addition to the actual distance between adjacent and consecutive macro-units in a pattern, the number of macro-units that appear on a substrate surface may also have an effect on whether the substrate surface may be perceived as three-dimensional. Without intending to be bound by any particular theory, in some embodiments, it may be preferable to have at least 10, 20, or 50 macro-units visible on a substrate.

It is to be appreciated that various embodiments of patterns may be disposed on various types of substrate surfaces that cause the macro-units and/or the substrate surfaces to exhibit a perceived three-dimensional appearance. As previously mentioned, the perceived three-dimensional appearance of the substrate surface can resemble protrusions and indentions indicative of threads in woven cloths, giving the substrate surface a cloth-like appearance. The patterns are created by printing color zones on a surface of a substrate. As mentioned above, embodiments of the patterns include a plurality of repeating shapes or macro-units, each macro-unit having three or more color zones. In some embodiments, all color zones are defined by printed colors. In other embodiments, one color zone may be defined by the substrate color. Based on the foregoing discussion, various guidelines may be applied to select pattern parameters to enhance the perceived appearance of a three-dimensional substrate surface upon which the pattern is disposed. In particular, the estimated distance of interaction, the number of color zones per macro-unit, the levels of contrast (i.e. ΔL*) between the color zones, the macro-unit size (i.e. characterized herein by the actual primary dimension), and the distances between adjacent macro-units may be selected based on the foregoing guidelines to enhance the perceived three-dimensional appearance of the substrate.

It is to be appreciated that additional pattern characteristics may further enhance the perceived three-dimensional appearance of the substrate surface. For example, some patterns may have anomalies or degree of randomness created by macro-units that differ slightly from each other in actual size, shape, maximum distance, L*, a* and/or b* values. Without intending to be bound by philosophical theory, it is believed that “perfection” in repeating shapes is seldom found in nature. Said differently, it is believed that the human brain will categorize a perfect repeating pattern as “artificial” as opposed to “natural.” Consequently, it is believed that a substrate with a repeating pattern including a plurality of macro-units such that at least some of the macro-units slightly differ from each other, will not only be perceived by a person as three-dimensional but also as more natural. In one embodiment, this slight degree of randomness or anomalies present on the macro-units may resemble imperfections of woven cloth, such as the result of having larger or smaller threads in certain areas. In some instances, pattern anomalies may be deliberately printed on the substrate. In another example, a substrate may include more than one pattern having macro-units of different actual sizes and/or shapes. By “random pattern” or “random repeating pattern,” it is meant a pattern having a plurality of macro-units such that at least some of the macro-units forming the pattern (for example at least 2, at least 5, at least 10 or even all the macro-units) differ from each other in a parameter chosen from at least one of actual primary dimension of the macro-units, shapes, maximum distance between macro-units, L*, a* and/or b* values of the color zone of the macro-units.

In one embodiment, a substrate may include a perceived three-dimensional pattern and at least a character graphic that be printed on the substrate. In one embodiment, the actual primary dimension of the character graphic is at least two, five or 10 times greater than the actual primary dimension of the macro-units forming the pattern. Without intending to be bound by any theory, it is believed that the presence of a such character graphic within the repeating pattern will direct the viewer's attention to the character graphic while allowing the viewer (consciously or unconsciously) to perceived the pattern. In a way, the character graphic helps “distract” the viewer's attention such that the viewer may not pay close attention to the repeating pattern while being cognizant of the pattern presence on the substrate.

In some embodiments, a printed substrate may be covered with an additional substrate to improve the overall appearance. For example, a printed substrate may be covered by an additional substrate having an opacity of less than 80% wherein the additional substrate softens the transitions between adjacent color zones. The additional substrate may cause the laminate to exhibit a softer appearance as well as provide a softer feel, thus combining visual and tactile stimuli.

Another characteristic that may further enhance the perceived three-dimensional appearance of the substrate surface may include two or more patterns that appear to be combinable to form another pattern. In addition, physical characteristics of the substrates, such as folding creases, in combination with the printed patterns may also enhance the perceived three-dimensional appearance of the substrate surface. In another scenario, a substrate may include a plurality of patterns that represent different three-dimensional features, such as different textures. In one example, a substrate may be printed with different patterns that represent different garment-like features, such as ribbed cuffs, collars, and/or woven edges or seams.

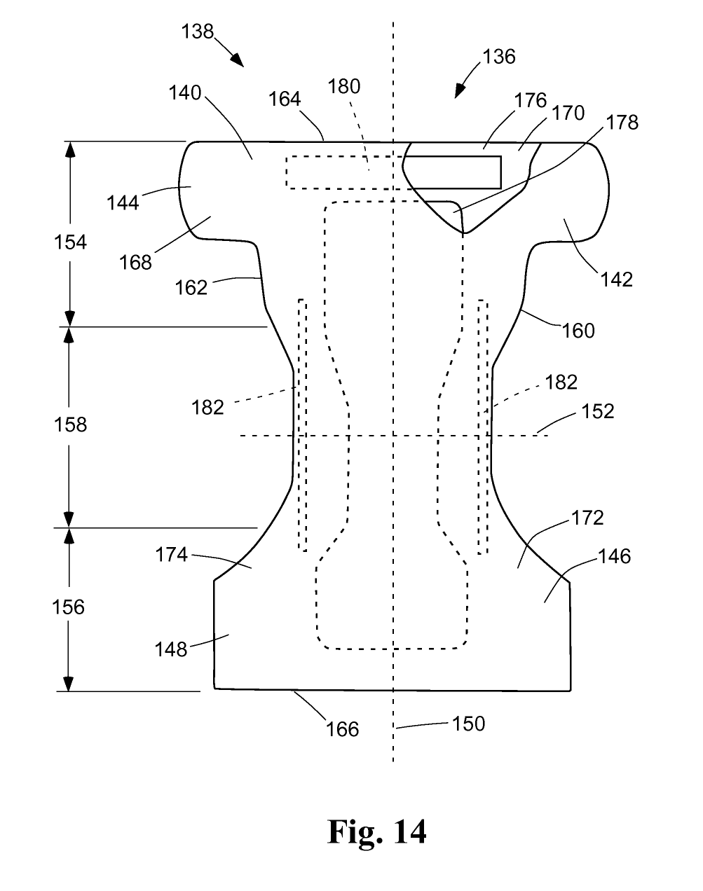

A number of different products that may utilize substrates with patterns printed thereon providing a desired perceived three-dimensional appearance are referred to above. For the purposes of a specific illustration, FIG. 14 shows one example of a disposable absorbent article 136 in the form of a diaper 138 that may include one or more substrates with patterns 100 disposed thereon in accordance with the above disclosure. In particular, FIG. 14 is a plan view of one embodiment of a diaper 138 including a chassis 140 shown in a flat, unfolded condition, with the portion of the diaper 138 that faces a wearer oriented towards the viewer. A portion of the chassis structure is cut-away in FIG. 14 to more clearly show the construction of and various features that may be included in embodiments of the diaper.

As shown in FIG. 14, the diaper 138 includes a chassis 140 having a first ear 142, a second ear 144, a third ear 146, and a fourth ear 148. To provide a frame of reference for the present discussion, the chassis is shown with a longitudinal axis 150 and a lateral axis 152. The chassis 140 is shown as having a first waist region 154, a second waist region 156, and a crotch region 158 disposed intermediate the first and second waist regions. The periphery of the diaper is defined by a pair of longitudinally extending side edges 160, 162; a first outer edge 164 extending laterally adjacent the first waist region 154; and a second outer edge 166 extending laterally adjacent the second waist region 156.

As shown in FIG. 14, the chassis 140 includes an inner, body-facing surface 168, and an outer, garment-facing surface 170. A portion of the chassis structure is cut-away in FIG. 14 to more clearly show the construction of and various features that may be included in the diaper. As shown in FIG. 14, the chassis 140 of the diaper 138 may include an outer covering layer 172 including a topsheet 174 and a backsheet 176. An absorbent core 178 may be disposed between a portion of the topsheet 174 and the backsheet 176. As discussed in more detail below, any one or more of the regions may be stretchable and may include an elastomeric material or laminate as described herein. As such, the diaper 138 may be configured to adapt to a specific wearer's anatomy upon application and to maintain coordination with the wearer's anatomy during wear.

In some instances, it may be desirable to provide a diaper, such as shown in FIG. 14, that including a backsheet, a topsheet, and/or side panels or ears having patterns disposed thereon that exhibit a three-dimensional or cloth-like appearance. When such components are stretchable, the patterns may be printed so as to appear three-dimensional in a contracted or a stretched state.

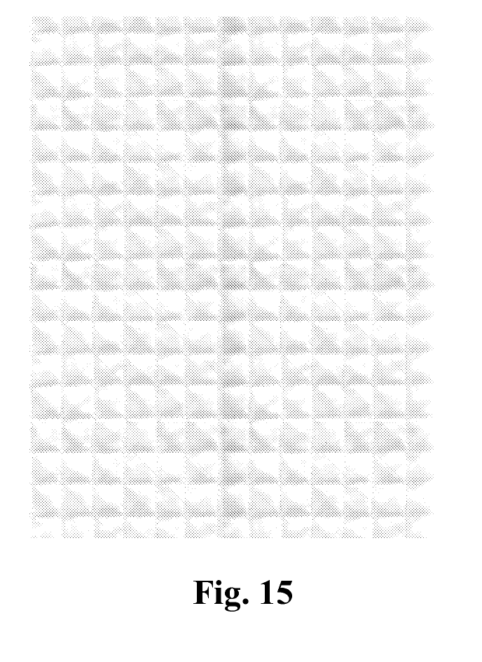

FIGS. 15-18 show various examples of patterns that may be applied to various diaper components, such as the backsheet, topsheet, absorbent core components, fastener elements, and/or ears or side panels.

The following provides a description of some of the various structural variations that may be included with various diaper and chassis embodiments.

As previously mentioned, the chassis 140 of the diaper 138 may include the backsheet 176, shown for example, in FIG. 14. In some embodiments, the backsheet is configured to prevent exudates absorbed and contained within the chassis from soiling articles that may contact the diaper, such as bedsheets and undergarments. Some embodiments of the backsheet may be fluid permeable, while other embodiments may be impervious to liquids (e.g., urine) and comprises a thin plastic film. In some embodiments, the plastic film includes a thermoplastic film having a thickness of about 0.012 mm (0.5 mil) to about 0.051 mm (2.0 mils). Some backsheet films may include those manufactured by Tredegar Industries Inc. of Terre Haute, Ind. and sold under the trade names X15306, X10962, and X10964. Other backsheet materials may include breathable materials that permit vapors to escape from the diaper while still preventing exudates from passing through the backsheet. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont and U.S. Pat. No. 5,865,823, issued on Feb. 2, 1999 to Curro, both of which are hereby incorporated by reference herein. Other breathable backsheets including nonwoven webs and apertured formed films are described in U.S. Pat. No. 5,571,096 issued to Dobrin et al. on Nov. 5, 1996; and U.S. Pat. No. 6,573,423 issued to Herrlein et al. on Jun. 3, 2003, which are all hereby incorporated by reference herein.

The backsheet 176, or any portion thereof, may be stretchable in one or more directions. In one embodiment, the backsheet may comprise a structural elastic-like film (“SELF”) web. Embodiments of SELF webs are more completely described in U.S. Pat. No. 5,518,801, entitled “Web Materials Exhibiting Elastic-Like Behavior,” which issued to Chappell et al. on May 21, 1996, U.S. Pat. No. 5,723,087, entitled “Web Materials Exhibiting Elastic-Like Behavior,” which issued to Chappell et al. on Mar. 3, 1998; U.S. Pat. No. 5,691,035, entitled “Web Materials Exhibiting Elastic-Like Behavior,” which issued to Chappell et al. on Nov. 25, 1997; U.S. Pat. No. 5,891,544, entitled “Web Materials Exhibiting Elastic-Like Behavior,” which issued to Chappell et al. on Apr. 6, 1999; U.S. Pat. No. 5,916,663, entitled “Web Materials Exhibiting Elastic-Like Behavior,” which issued to Chappell et al. on Jun. 29, 1999; and U.S. Pat. No. 6,027,483, entitled “Web Materials Exhibiting Elastic-Like Behavior,” which issued to Chappell et al. on Feb. 22, 2000, which are all hereby incorporated by reference herein. In some embodiments, the backsheet may comprise elastomeric films, foams, strands, nonwovens, or combinations of these or other suitable materials with nonwovens or synthetic films. Additional embodiments include backsheets that comprise a stretch nonwoven material; an elastomeric film in combination with an extensible nonwoven; an elastomeric nonwoven in combination with an extensible film; and/or combinations thereof. Details on such backsheet embodiments are more completely described in U.S. non-provisional patent application entitled “Biaxially Stretchable Outer Cover for an Absorbent Article,” filed on Nov. 15, 2006 with Express Mail No. EV916939625US and further identified by attorney docket number 10643 and U.S. application Ser. No. 11/599,829; U.S. non-provisional patent application entitled “Disposable Wearable Articles with Anchoring Systems,” filed on Nov. 15, 2006 with Express Mail No. EV916939648US and further identified by attorney docket number 10628Q and U.S. application Ser. No. 11/599,851; and U.S. non-provisional patent application entitled “Absorbent Article having an Anchored Core Assembly,” filed on Nov. 15, 2006 with Express Mail No. EV916939634US and further identified by attorney docket number 10432MΩ and U.S. application Ser. No. 11/599,862, which are all hereby incorporated by reference herein.

The backsheet 176 may be joined with the topsheet 174, the absorbent core 178, and/or other elements of the diaper 138 in various ways. For example, the backsheet may be connected with a uniform continuous layer of adhesive, a patterned layer of adhesive, or an array of separate lines, spirals, or spots of adhesive. One embodiment utilizes an open pattern network of filaments of adhesive as disclosed in U.S. Pat. No. 4,573,986, entitled “Disposable Waste-Containment Garment,” which issued to Minetola et al. on Mar. 4, 1986, which is hereby incorporated by reference herein. Other embodiments utilize several lines of adhesive filaments which are swirled into a spiral pattern, as is illustrated by the apparatus and methods shown in U.S. Pat. No. 3,911,173, issued to Sprague, Jr. on Oct. 7, 1975; U.S. Pat. No. 4,785,996, issued to Ziecker, et al. on Nov. 22, 1988; and U.S. Pat. No. 4,842,666 issued to Werenicz on Jun. 27, 1989, which are all hereby incorporated by reference herein. Adhesives may include those manufactured by H. B. Fuller Company of St. Paul, Minn. and marketed as HL-1620 and HL-1358-XZP. In some embodiments, the backsheet is connected with heat bonds, pressure bonds, ultrasonic bonds, dynamic mechanical bonds, or any other suitable attachment means or a combination thereof.

The topsheet 174 may be joined to the backsheet 176, the absorbent core 178, and/or other elements of the diaper 138 in various ways. For example, the topsheet 174 may be connected in ways described above with respect to joining the backsheet 176 to other elements of the diaper 138. In one embodiment, the topsheet 174 and the backsheet 176 are joined directly to each other along the outer edge of the chassis. In another embodiment, the topsheet and the backsheet are joined directly to each other in some locations and are indirectly joined together in other locations. Other topsheet and backsheet connection configurations are described in more detail in U.S. provisional patent application No. 60/811,700, entitled “Absorbent Article Having a Multifunctional Containment Member,” filed on Jun. 7, 2006, which is hereby incorporated by reference herein.

The topsheet 140 may be constructed to be compliant, soft feeling, and non-irritating to the wearer's skin. Further, all or at least a portion of the topsheet 140 may be liquid pervious, permitting liquid to readily penetrate therethrough. As such, the topsheet may be manufactured from a wide range of materials, such as porous foams; reticulated foams; apertured nonwovens or plastic films; or woven or nonwoven webs of natural fibers (e.g., wood or cotton fibers), synthetic fibers (e.g., polyester or polypropylene fibers), or a combination of natural and synthetic fibers. If the absorbent assemblies include fibers, the fibers may be spunbonded, carded, wet-laid, meltblown, hydroentangled, or otherwise processed as is known in the art. One example of a topsheet including a web of staple length polypropylene fibers is manufactured by Veratec, Inc., a Division of International Paper Company, of Walpole, Mass. under the designation P-8.

Examples of formed film topsheets are described in U.S. Pat. No. 3,929,135, entitled “Absorptive Structures Having Tapered Capillaries,” which issued to Thompson on Dec. 30, 1975; U.S. Pat. No. 4,324,246, entitled “Disposable Absorbent Article Having A Stain Resistant Topsheet,” which issued to Mullane, et al. on Apr. 13, 1982; U.S. Pat. No. 4,342,314, entitled “Resilient Plastic Web Exhibiting Fiber-Like Properties,” which issued to Radel, et al. on Aug. 3, 1982; U.S. Pat. No. 4,463,045, entitled “Macroscopically Expanded Three-Dimensional Plastic Web Exhibiting Non-Glossy Visible Surface and Cloth-Like Tactile Impression,” which issued to Ahr, et al. on Jul. 31, 1984; and U.S. Pat. No. 5,006,394, entitled “Multilayer Polymeric Film,” which issued to Baird on Apr. 9, 1991, all of which are hereby incorporated by reference herein. Other topsheets may be made in accordance with U.S. Pat. Nos. 4,609,518 and 4,629,643, which issued to Curro et al. on Sep. 2, 1986, and Dec. 16, 1986, respectively, both of which are hereby incorporated by reference herein. Such formed films are available from The Procter & Gamble Company of Cincinnati, Ohio as “DRI-WEAVE” and from Tredegar Corporation of Terre Haute, Ind. as “CLIFF-T.”

In some embodiments, the topsheet 174 is made of a hydrophobic material or is treated to be hydrophobic in order to isolate the wearer's skin from liquids contained in the absorbent core. If the topsheet is made of a hydrophobic material, at least the upper surface of the topsheet may be treated to be hydrophilic so that liquids will transfer through the topsheet more rapidly. This diminishes the likelihood that body exudates will flow off the topsheet rather than being drawn through the topsheet and being absorbed by the absorbent core. The topsheet can be rendered hydrophilic by treating it with a surfactant or by incorporating a surfactant into the topsheet. Suitable methods for treating the topsheet with a surfactant include spraying the topsheet material with the surfactant and immersing the material into the surfactant. A more detailed discussion of such a treatment and hydrophilicity is contained in U.S. Pat. No. 4,988,344, entitled “Absorbent Articles with Multiple Layer Absorbent Layers,” which issued to Reising, et al. on Jan. 29, 1991, and U.S. Pat. No. 4,988,345, entitled “Absorbent Articles with Rapid Acquiring Absorbent Cores,” which issued to Reising on Jan. 29, 1991, all of which are hereby incorporated by reference herein. A more detailed discussion of some methods for incorporating surfactant in the topsheet can be found in U.S. Statutory Invention Registration No. H1670, which was published on Jul. 1, 1997, in the names of Aziz et al., all of which are hereby incorporated by reference herein.

In some embodiments, the topsheet 174 may include an apertured web or film that is hydrophobic. This may be accomplished eliminating the hydrophilizing treatment step from the production process and/or applying a hydrophobic treatment to the topsheet, such as a polytetrafluoroethylene compound like SCOTCHGUARD or a hydrophobic lotion composition, as described below. In such embodiments, the apertures may be large enough to allow the penetration of aqueous fluids like urine without significant resistance. A more detailed discussion of various apertured topsheets can be found in U.S. Pat. No. 5,342,338, entitled “Disposable Absorbent Article for Low-Viscosity Fecal Material,” which issued to Roe on Aug. 30, 1994; U.S. Pat. No. 5,941,864, entitled “Disposable Absorbent Article having Improved Fecal Storage,” which issued to Roe on Aug. 24, 1999; U.S. Pat. No. 6,010,491, entitled “Viscous Fluid Bodily Waste Management Article,” which issued to Roe et al. on Jan. 4, 2000; and U.S. Pat. No. 6,414,215, entitled “Disposable Absorbent Article having Capacity to Store Low-Viscosity Fecal Material,” which issued to Roe on Jul. 2, 2002, all of which are hereby incorporated by referenced herein.

Any portion of the topsheet 174 may be coated with a lotion, such as topsheets described in U.S. Pat. No. 5,607,760, entitled “Disposable Absorbent Article Having A Lotioned Topsheet Containing an Emollient and a Polyol Polyester Immobilizing Agent,” which issued to Roe on Mar. 4, 1997; U.S. Pat. No. 5,609,587, entitled “Diaper Having A Lotion Topsheet Comprising A Liquid Polyol Polyester Emollient And An Immobilizing Agent,” which issued to Roe on Mar. 11, 1997; U.S. Pat. No. 5,635,191, entitled “Diaper Having A Lotioned Topsheet Containing A Polysiloxane Emollient,” which issued to Roe et al. on Jun. 3, 1997; U.S. Pat. No. 5,643,588, entitled “Diaper Having A Lotioned Topsheet,” which issued to Roe et al. on Jul. 1, 1997; and U.S. Pat. No. 6,498,284, entitled “Disposable Absorbent Article with a Skin Care Composition on an Apertured Top Sheet,” which issued to Roe on Dec. 24, 2002, all of which are hereby incorporated by reference herein. The lotion may function alone or in combination with another agent as the hydrophobizing treatment described above. The topsheet may also include or be treated with antibacterial agents, some examples of which are disclosed in PCT Publication No. WO 95/24173 entitled “Absorbent Articles Containing Antibacterial Agents in the Topsheet For Odor Control,” which was published on Sep. 14, 1995, in the name of Theresa Johnson, which is hereby incorporated by reference herein. Further, the topsheet, the backsheet, or any portion of the topsheet or backsheet may be embossed and/or matte finished to provide a more cloth like appearance.

Embodiments of the absorbent article may also include pockets for receiving and containing waste, spacers which provide voids for waste, barriers for limiting the movement of waste in the article, compartments or voids which accept and contain waste materials deposited in the diaper, and the like, or any combinations thereof. Examples of pockets and spacers for use in absorbent products are described in U.S. Pat. No. 5,514,121 issued to Roe et al. on May 7, 1996, entitled “Diaper Having Expulsive Spacer”; U.S. Pat. No. 5,171,236 issued to Dreier et al on Dec. 15, 1992, entitled “Disposable Absorbent Article Having Core Spacers”; U.S. Pat. No. 5,397,318 issued to Dreier on Mar. 14, 1995, entitled “Absorbent Article Having A Pocket Cuff”; U.S. Pat. No. 5,540,671 issued to Dreier on Jul. 30, 1996, entitled “Absorbent Article Having A Pocket Cuff With An Apex”; and PCT Application WO 93/25172 published Dec. 3, 1993, entitled “Spacers For Use In Hygienic Absorbent Articles And Disposable Absorbent Articles Having Such Spacer”; and U.S. Pat. No. 5,306,266, entitled “Flexible Spacers For Use In Disposable Absorbent Articles”, issued to Freeland on Apr. 26, 1994, which are all hereby incorporated by reference herein. Examples of compartments or voids are disclosed in U.S. Pat. No. 4,968,312, entitled “Disposable Fecal Compartmenting Diaper”, issued to Khan on Nov. 6, 1990; U.S. Pat. No. 4,990,147, entitled “Absorbent Article With Elastic Liner For Waste Material Isolation”, issued to Freeland on Feb. 5, 1991; U.S. Pat. No. 5,62,840, entitled “Disposable Diapers”, issued to Holt et al on Nov. 5, 1991; U.S. Pat. No. 6,482,191 entitled “Elasticated Topsheet with an Elongate Slit Opening,” issued to Roe et al. on Nov. 19, 2002; and U.S. Pat. No. 5,269,755 entitled “Trisection Topsheets For Disposable Absorbent Articles And Disposable Absorbent Articles Having Such Trisection Topsheets”, issued to Freeland et al. on Dec. 14, 1993, which are all hereby incorporated by reference herein. Examples of suitable transverse barriers are described in U.S. Pat. No. 5,554,142 entitled “Absorbent Article Having Multiple Effective Height Transverse Partition” issued Sep. 10, 1996 in the name of Dreier et al.; PCT Patent WO 94/14395 entitled “Absorbent Article Having An Upstanding Transverse Partition” published Jul. 7, 1994 in the name of Freeland, et al., and U.S. Pat. No. 5,653,703 Absorbent Article Having Angular Upstanding Transverse Partition, issued Aug. 5, 1997 to Roe, et al., which are all hereby incorporated by reference herein. All of the above-cited references are hereby incorporated by reference herein. In addition to or in place of the voids, pockets and barriers, described above, embodiments of the absorbent article may also include a waste management element capable of effectively and efficiently accepting, storing and/or immobilizing viscous fluid bodily waste, such as runny feces, such as described in U.S. Pat. No. 6,010,491 issued to Roe et al. on Jan. 4, 2000, which is hereby incorporated by reference herein.

The absorbent core 178 may include absorbent material that is generally compressible, conformable, non-irritating to the wearer's skin, and capable of absorbing and retaining liquids such as urine and other body exudates. The absorbent core 178 can also be manufactured in a wide variety of sizes and shapes (e.g., rectangular, hourglass, T-shaped, asymmetric, etc.). The absorbent core may also include a wide variety of liquid-absorbent materials commonly used in disposable diapers and other absorbent articles. In one example, the absorbent core includes comminuted wood pulp, which is generally referred to as airfelt. Examples of other absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

It is to be appreciated that the configuration and construction of the absorbent core 178 may be varied (e.g., the absorbent core(s) or other absorbent structure(s) may have varying caliper zones, a hydrophilic gradient, a superabsorbent gradient, or lower average density and lower average basis weight acquisition zones; or may comprise one or more layers or structures).

Exemplary absorbent structures are described in U.S. Pat. No. 4,610,678, entitled “High-Density Absorbent Structures,” which issued to Weisman et al. on Sep. 9, 1986; U.S. Pat. No. 4,673,402, entitled “Absorbent Articles With Dual-Layered Cores,” which issued to Weisman et al. on Jun. 16, 1987; U.S. Pat. No. 4,834,735, entitled “High Density Absorbent Members Having Lower Density and Lower Basis Weight Acquisition Zones,” which issued to Alemany et al. on May 30, 1989; U.S. Pat. No. 4,888,231, entitled “Absorbent Core Having A Dusting Layer,” which issued to Angstadt on Dec. 19, 1989; U.S. Pat. No. 5,137,537, entitled “Absorbent Structure Containing Individualized, Polycarboxylic Acid Crosslinked Wood Pulp Cellulose Fibers,” which issued to Herron et al. on Aug. 11, 1992; U.S. Pat. No. 5,147,345, entitled “High Efficiency Absorbent Articles For Incontinence Management,” which issued to Young et al. on Sep. 15, 1992; U.S. Pat. No. 5,342,338, entitled “Disposable Absorbent Article For Low-Viscosity Fecal Material,” issued to Roe on Aug. 30, 1994; U.S. Pat. No. 5,260,345, entitled “Absorbent Foam Materials For Aqueous Body Fluids and Absorbent Articles Containing Such Materials,” which issued to DesMarais et al. on Nov. 9, 1993; U.S. Pat. No. 5,387,207, entitled “Thin-Until-Wet Absorbent Foam Materials For Aqueous Body Fluids And Process For Making Same,” which issued to Dyer et al. on Feb. 7, 1995; and U.S. Pat. No. 5,650,222, entitled “Absorbent Foam Materials For Aqueous Fluids Made From high Internal Phase Emulsions Having Very High Water-To-Oil Ratios,” which issued to DesMarais et al. on Jul. 22, 1997, all of which are hereby incorporated by reference herein.

The absorbent core 178 may also have a multiple layered construction. A more detailed discussion of various types of multi-layered absorbent cores can be found in U.S. Pat. No. 5,669,894, entitled “Absorbent Members for Body Fluids having Good Wet Integrity and Relatively High Concentrations of Hydrogel-forming Absorbent Polymer,” issued to Goldman et al. on Sep. 23, 1997; U.S. Pat. No. 6,441,266, entitled “Absorbent Members for Body Fluids using Hydrogel-forming Absorbent Polymer,” issued to Dyer et al. on Aug. 26, 2002; U.S. Pat. No. 5,562,646, entitled “Absorbent Members for Body Fluids having Good Wet Integrity and Relatively High Concentrations of Hydrogel-forming Absorbent Polymer having High Porosity,” issued to Goldman et al. on Oct. 10, 1996; European Pat. No. EP0565606B1, published on Mar. 8, 1995; U.S. Pat. Publication No. 2004/0162536A1 published Aug. 19, 2004; U.S. Pat. Publication No. 2004/0167486A1 published on Aug. 26, 2004; and PCT Publication No. WO 2006/015141 published on Feb. 9, 2006, which are all hereby incorporated by reference herein. In some embodiments, the absorbent article includes an absorbent core that is stretchable. In such a configuration, the absorbent core may be adapted to extend along with other materials of the chassis in longitudinal and/or lateral directions. The absorbent core can also be connected with the other components of the chassis various ways. For example, the diaper may include a “floating core” configuration or a “bucket” configuration wherein the diaper includes an anchoring system that can be configured to collect forces tending to move the article on the wearer. Such an anchoring system can also be configured to anchor itself to a body of a wearer by contacting various parts of the body. In this way, the anchoring system can balance the collected moving forces with holding forces obtained from the anchoring. By balancing the collected moving forces with the obtained holding forces, the anchoring system can at least assist in holding the disposable wearable absorbent article in place on a wearer. A more detailed discussion of various floating and/or bucket core configurations can be found in U.S. provisional patent application No. 60/811,700, entitled “Absorbent Article Having a Multifunctional Containment Member,” filed on Jun. 7, 2006; U.S. non-provisional patent application entitled “Disposable Wearable Articles with Anchoring Systems,” filed on Nov. 15, 2006 with Express Mail No. EV916939648US and further identified by attorney docket number 10628Q and U.S. application Ser. No. 11/599,851; and U.S. non-provisional patent application entitled “Absorbent Article having an Anchored Core Assembly,” filed on Nov. 15, 2006 with Express Mail No. EV916939634US and further identified by attorney docket number 10432MΩ and U.S. application Ser. No. 11/599,862, which are all hereby incorporated by reference herein.

The diaper 138 may also include at least one elastic waist feature 180, shown for example in FIG. 14, which may provide improved fit and waste containment. The elastic waist feature 180 may be configured to elastically expand and contract to dynamically fit the wearer's waist. The elastic waist feature 180 may extend at least longitudinally outwardly from the absorbent core 178 and generally form at least a portion of the first and/or second outer edges 164, 166 of the diaper 138. In addition, the elastic waist feature may extend laterally to include the ears. While the elastic waist feature 180 or any constituent elements thereof may comprise one or more separate elements affixed to the diaper, the elastic waist feature may be constructed as an extension of other elements of the diaper, such as the backsheet 176, the topsheet 174, or both the backsheet and the topsheet. In addition, the elastic waist feature 180 may be disposed on the outer, garment-facing surface 170 of the chassis 140; the inner, body-facing surface 168; or between the inner and outer facing surfaces.

The elastic waist feature 180 may be constructed in a number of different configurations including those described in U.S. Pat. No. 4,515,595, which issued to Kievit et al. on May 7, 1985; U.S. Pat. No. 4,710,189, which issued to Lasch on Dec. 1, 1987; U.S. Pat. No. 5,151,092, which issued to Buell on Sep. 9, 1992; and U.S. Pat. No. 5,221,274, which issued to Buell on Jun. 22, 1993, all of which are hereby incorporated by reference herein. Other waist configurations may include waistcap features such as those described in U.S. Pat. No. 5,026,364, which issued to Robertson on Jun. 25, 1991 and U.S. Pat. No. 4,816,025, which issued to Foreman on Mar. 28, 1989, both of which are hereby incorporated by reference herein.

Although the first and second ears 142, 144 as well as the third and fourth ears 146, 148 shown in FIG. 14 are illustrated as being integrally formed with the chassis 140, it is to be appreciated that other embodiments may include ears that are discrete elements connected with the chassis. In some embodiments, the ears are configured to be stretchable, and in some embodiments, it may be preferable to have elastically stretchable ears. As discussed in more detail below, the ears may also include one or more fastener elements 150 adapted to releasably connect with each other and/or other fastener elements on the chassis. A more detailed discussion of stretchable ears can be found in U.S. Pat. No. 4,857,067, entitled “Disposable Diaper Having Shirred Ears” issued to Wood, et al. on Aug. 15, 1989; U.S. Pat. No. 5,151,092 issued to Buell et al. on Sep. 29, 1992; U.S. Pat. No. 5,674,216 issued to Buell et al. on Oct. 7, 1997; U.S. Pat. No. 6,677,258 issued to Carroll et al. on Jan. 13, 2004; U.S. Pat. No. 4,381,781 issued to Sciaraffa, et al. on May 3, 1983; U.S. Pat. No. 5,580,411 entitled “Zero Scrap Method For Manufacturing Side Panels For Absorbent Articles” issued to Nease, et al. on Dec. 3, 1996; and U.S. Pat. No. 6,004,306 entitled “Absorbent Article With Multi-Directional Extensible Side Panels” issued to Robles et al. on Dec. 21, 1999, which are all hereby incorporated by reference herein. The ears may also include various geometries and arrangements of stretch zones or elements, such as discussed in U.S. Pat. Publication No. US2005/0215972A1 published on Sep. 29, 2005, and U.S. Pat. Publication No. US2005/0215973A1 published on Sep. 29, 2005, which are all hereby incorporated by reference herein.

As shown in FIG. 14, the diaper 138 may include leg cuffs 182 that may provide improved containment of liquids and other body exudates. In particular, elastic gasketing leg cuffs can provide a sealing effect around the wearer's thighs to prevent leakage. It is to be appreciated that when the diaper is worn, the leg cuffs may be placed in contact with the wearer's thighs, and the extent of that contact and contact pressure may be determined in part by the orientation of diaper on the body of the wearer. The leg cuffs 182 may be disposed in various ways on the diaper 102. For example, the leg cuffs 182 may be disposed on the outer, garment-facing surface 170 of the chassis 138; the inner, body-facing surface 168; or between the inner and outer facing surfaces. Leg cuffs 182 may also be referred to as leg bands, side flaps, barrier cuffs, or elastic cuffs. U.S. Pat. No. 3,860,003, which is hereby incorporated by reference herein, describes a disposable diaper that provides a contractible leg opening having a side flap and one or more elastic members to provide an elasticized leg cuff (a gasketing cuff). U.S. Pat. Nos. 4,808,178 and 4,909,803, issued to Aziz et al. on Feb. 28, 1989, and Mar. 20, 1990, respectively, which are both hereby incorporated by reference herein, describe disposable diapers having “stand-up” elasticized flaps (barrier cuffs) which improve the containment of the leg regions. U.S. Pat. Nos. 4,695,278 and 4,795,454, issued to Lawson on Sep. 22, 1987, and to Dragoo on Jan. 3, 1989, respectively, which are both hereby incorporated by reference herein, describe disposable diapers having dual cuffs, including gasketing cuffs and barrier cuffs. In some embodiments, it may be desirable to treat all or a portion of the leg cuffs with a lotion, as described above. In addition to leg cuffs, diaper can also include an elastic gasketing cuff with one or more elastic strands positioned outboard of the barrier cuff. To improve waste containment, the leg cuffs may be treated with a hydrophobic surface coating, such as described in U.S. Pat. Publication No. 20060189956A1, entitled “Hydrophobic Surface Coated Light-Weight Nonwoven Laminates for Use in Absorbent Articles,” published on Aug. 24, 2006, which is hereby incorporated by reference herein.

The diaper 138 may be provided in the form of a pant-type diaper or may alternatively be provided with a re-closable fastening system, which may include fastener elements in various locations to help secure the diaper in position on the wearer. For example, fastener elements may be located on the first and second ears and may be adapted to releasably connect with one or more corresponding fastening elements located in the second waist region.

It is to be appreciated that various types of fastening elements may be used with the diaper. In one example, the fastening elements include hook & loop fasteners, such as those available from 3M or Velcro Industries. In other examples, the fastening elements include adhesives and/or tap tabs, while others are configured as a macrofastener or hook (e.g., a MACRO or “button-like” fastener). Some exemplary fastening elements and systems are disclosed in U.S. Pat. No. 3,848,594, entitled “Tape Fastening System for Disposable Diaper,” which issued to Buell on Nov. 19, 1974; U.S. Pat. No. 4,662,875, entitled “Absorbent Article,” which issued to Hirotsu et al. on May 5, 1987; U.S. Pat. No. 4,846,815, entitled “Disposable Diaper Having An Improved Fastening Device,” which issued to Scripps on Jul. 11, 1989; U.S. Pat. No. 4,894,060, entitled “Disposable Diaper With Improved Hook Fastener Portion,” which issued to Nestegard on Jan. 16, 1990; U.S. Pat. No. 4,946,527, entitled “Pressure-Sensitive Adhesive Fastener And Method of Making Same,” which issued to Battrell on Aug. 7, 1990; and U.S. Pat. No. 5,151,092, issued to Buell on Sep. 29, 1992; and U.S. Pat. No. 5,221,274, which issued to Buell on Jun. 22, 1993, which are all hereby incorporated by reference herein. Additional examples of fasteners and/or fastening elements are discussed in U.S. Pat. Nos. 6,251,097 and 6,432,098; U.S. patent application Ser. No. 11/240,943, entitled, “Anti-Pop Open Macrofasteners” filed on Sep. 30, 2005; and U.S. patent application Ser. No. 11/240,838, entitled, “A Fastening System Having Multiple Engagement Orientations”, filed on Sep. 30, 2005, which are all hereby incorporated by reference herein. Other fastening systems are described in more detail in U.S. Pat. No. 5,595,567 issued to King et al. on Jan. 21, 1997 and U.S. Pat. No. 5,624,427 issued to Bergman et al. on Apr. 29, 1997, both of which are entitled “Nonwoven Female Component For Refastenable Fastening Device.” Yet other fastening systems are described in U.S. Pat. Nos. 5,735,840 and 5,928,212, both of which issued to Kline et al. and are entitled “Disposable Diaper With Integral Backsheet Landing Zone,” which are both hereby incorporated by reference herein. The fastening system may also provide a means for holding the article in a disposal configuration as disclosed in U.S. Pat. No. 4,963,140, which issued to Robertson et al. on Oct. 16, 1990, which is hereby incorporated by reference herein.

It is also to be appreciated that diapers 138 according the present disclosure may be constructed with various types of the previously described materials that allow the entire chassis 140 or portions of the chassis, such as the ears 142, 144, 146, 148, crotch region 158, and/or waist regions 154, 156 to stretch. It is to be appreciated that the entire chassis or portions of the chassis can be configured to stretch in longitudinal directions, lateral directions, or both (i.e. biaxial stretch). In some embodiments, the chassis may include regions of longitudinal stretch, regions of lateral stretch, and/or regions of biaxial stretch. For example, in some embodiments, the entire length of the crotch region 158 is adapted to stretch in longitudinal and/or lateral directions. In other embodiments, opposing end regions of the crotch region 158 is the only portion of the chassis 140 that is longitudinally and/or laterally stretchable. In yet other embodiments, central or proximal regions of the crotch region are the only portions of the chassis 140 that are longitudinally and/or laterally stretchable. In such example configurations, the crotch region or sub-regions thereof may comprise a different material than that of the remainder of the chassis 140, may have been subjected to a different treatment (e.g. SELFing, mechanical ringrolling), or a combination thereof. References disclosing structural elastic-like film (“SELF”) materials are discussed above. The chassis may also be constructed with a “zero strain” stretch laminate. Zero strain stretch laminates can be made by bonding an elastomer to a nonwoven while both are in an unstrained state. A more detailed discussion of zero strain laminates can be found in U.S. Pat. No. 5,156,793, entitled “Method for Incrementally Stretching Zero Strain Stretch Laminate Web in a Non-uniform Manner to Impart a Varying Degree of Elasticity Thereto,” issued to Buell et al. on Oct. 20, 1992, which is hereby incorporated by reference herein. In another example, the chassis may be constructed with “live stretch,” which may include stretching elastic and bonding the stretched elastic to a nonwoven. After bonding the stretched elastic is released causing it to contract, resulting in a “corrugated” nonwoven. A more detailed discussion of “live stretch” can be found in U.S. Pat. No. 4,720,415 to Vander Wielen, et al., issued Jan. 19, 1988 and U.S. Pat. No. 7,028,735 to Schneider et al. issued on Apr. 18, 2006, which are hereby incorporated by reference herein.

As previously mentioned, various repeating patterns can be printed on various types of substrates in order to provide the substrate with a perceived three-dimensional pattern, which may cause a visible surface of the substrate to exhibit a three-dimensional appearance. The following tables provide L* data measured from different patterns, having macro-units with various numbers of zones, which were printed on different substrates.

With reference to the Tables 1-12 below, L*1 corresponds with the L* measured in color zone 1, L*2 corresponds with the L* value measured in zone 2, L*3 corresponds with the L* value measured in zone 3, L*4 corresponds with the L* measured in color zone 4, L*5 corresponds with the L* measured in color zone 5, and L*6 corresponds with the L* measured in color zone 6. The L* values shown in Tables 1-12 were measured according to the L* measurement procedure described below. Further, the values of ΔL* in Tables 1-12 are defined as follows:

ΔL*₁₂=L*1−L*2;

ΔL*₁₃=L*1−L*3;

ΔL*₂₃=L*2−L*3;

ΔL*₃₄=L*3−L*4;

ΔL*₄₅=L*4−L*5; and

ΔL*₅₆=L*5−L*6.

### Test Sample 1

Test Sample 1 includes a circular-shaped macro-unit with a 1.5 mm diameter printed on a nonwoven substrate and having three color zones, wherein the lightest color zone is defined by the color of the nonwoven substrate and the other two color zones are printed on the nonwoven substrate. The nonwoven substrate of Test Sample 1 is a 27 gsm carded polypropylene. For reference, the circular shape is generally represented by a circular-shaped macro-unit shown in FIG. 8 as having a Upd of 1.5 mm and 3 color zones.

TABLE 1
L* Measurements from Test Sample 1

| L*₁ | L*₂ | L*₃ | ΔL*₁₂ | ΔL*₂₃ | ΔL*₁₃ |
|---|---|---|---|---|---|
| 88.1 | 83.4 | 77.2 | 4.7 | 6.2 | 10.9 |

### Test Sample 2

Test Sample 2 includes a circular-shaped macro-unit with a 1.5 mm diameter printed on a nonwoven-film laminate substrate and having three color zones, wherein the lightest color zone is defined by the color of the nonwoven-film substrate and the other two color zones are printed on the nonwoven-film substrate. Specifically, the macro-unit is printed onto a nonwoven fabric, which is adhered to a film substrate. The nonwoven-film substrate of Test Sample 2 includes a 27 gsm carded polypropylene nonwoven adhered to a 18 gsm polypropylene/polyethylene (PP/PE) film. For reference, the circular shape is generally represented by a circular-shaped macro-unit shown in FIG. 8 as having a Upd of 1.5 mm and 3 color zones.

TABLE 2
L* Measurements from Test Sample 2

| | | | | | |
|---|---|---|---|---|---|
| L*₁ | L*₂ | L*₃ | ΔL*₁₂ | ΔL*₂₃ | ΔL*₁₃ |

| | | | | | |
|---|---|---|---|---|---|
| 84.4 | 78.1 | 72.6 | 6.3 | 5.5 | 11.8 |

### Test Sample 3

Test Sample 3 includes a circular-shaped macro-unit with a 1.5 mm diameter printed on a film substrate and having three color zones, wherein the lightest color zone is defined by the color of the film substrate and the other two color zones are printed on the film substrate. The film substrate of Test Sample 3 is a 18 gsm polypropylene/polyethylene (PP/PE) film. For reference, the circular shape is generally represented by a circular-shaped macro-unit shown in FIG. 8 as having a Upd of 1.5 mm and 3 color zones.

TABLE 3
L* Measurements from Test Sample 3

| L*₁ | L*₂ | L*₃ | ΔL*₁₂ | ΔL*₂₃ | ΔL*₁₃ |
|---|---|---|---|---|---|
| 98.7 | 90.6 | 82.4 | 8.1 | 8.2 | 16.3 |

As illustrated by the data in Tables 1-3, the macro-units of Test Samples 1, 2, and 3 have zones with L* values that fall within the following criteria described above:

L1>L2>L3,

3≦(L1−L3), and

2≦(L1−L2)≦10.

### Test Sample 4

Test Sample 4 includes a circular-shaped macro-unit with a 3.5 mm diameter printed on a nonwoven substrate and having five color zones, wherein the lightest color zone is defined by the color of the nonwoven substrate and the other four color zones are printed on the nonwoven substrate. The nonwoven substrate of Test Sample 4 is a 27 gsm carded polypropylene. For reference, the circular shape is generally represented by a circular-shaped macro-unit shown in FIG. 8 as having a Upd of 3.5 mm and 5 color zones.

TABLE 4
L* Measurements from Test Sample 4

| L*₁ | L*₂ | L*₃ | L*₄ | L*₅ | ΔL*₁₂ | ΔL*₂₃ | ΔL*₃₄ | ΔL*₄₅ |
|---|---|---|---|---|---|---|---|---|
| 94.3 | 89.4 | 86.3 | 83.0 | 79.1 | 4.9 | 3.1 | 3.3 | 3.9 |

### Test Sample 5

Test Sample 5 includes a circular-shaped macro-unit with a 3.5 mm diameter printed on a nonwoven-film laminate substrate and having five color zones, wherein the lightest color zone is defined by the color of the nonwoven-film substrate and the other four color zones are printed on the nonwoven-film substrate. Specifically, the macro-unit is printed onto a nonwoven fabric, which is adhered to a film substrate. The nonwoven-film substrate of Test Sample 5 is a 27 gsm carded polypropylene nonwoven adhered to a 18 gsm polypropylene/polyethylene (PP/PE) film. For reference, the circular shape is generally represented by a circular-shaped macro-unit shown in FIG. 8 as having a Upd of 3.5 mm and 5 color zones.

TABLE 5
L* Measurements from Test Sample 5

| L*₁ | L*₂ | L*₃ | L*₄ | L*₅ | ΔL*₁₂ | ΔL*₂₃ | ΔL*₃₄ | ΔL*₄₅ |
|---|---|---|---|---|---|---|---|---|
| 90.8 | 84.1 | 79.2 | 73.4 | 67.6 | 6.7 | 5.1 | 5.8 | 5.8 |

### Test Sample 6

Test Sample 6 includes a circular-shaped macro-unit with a 3.5 mm diameter printed on a film substrate and having five color zones, wherein the lightest color zone is defined by the color of the film substrate and the other four color zones are printed on the film substrate. The film substrate of Test Sample 6 is a 18 gsm polypropylene/polyethylene (PP/PE) film. For reference, the circular shape is generally represented by a circular-shaped macro-unit shown in FIG. 8 as having a Upd of 3.5 mm and 5 color zones.

TABLE 6
L* Measurements from Test Sample 6

| L*₁ | L*₂ | L*₃ | L*₄ | L*₅ | ΔL*₁₂ | ΔL*₂₃ | ΔL*₃₄ | ΔL*₄₅ |
|---|---|---|---|---|---|---|---|---|
| 98.7 | 94.9 | 87.7 | 80.0 | 75.8 | 3.8 | 7.2 | 7.7 | 4.2 |

As illustrated by the data in Tables 4-6, the macro-units of Test Samples 4, 5, and 6 have zones with L* values that fall within the following criteria described above:

L1>L2>L3>L4>L5,

2≦(L1−L2)≦10,

2≦(L2−L3),

2≦(L3−L4), and

2≦(L4−L5).

### Test Sample 7

Test Sample 7 includes a circular-shaped macro-unit with a 7.5 mm diameter printed on a nonwoven substrate and having six color zones, wherein the lightest color zone is defined by the color of the nonwoven substrate and the other five color zones are printed on the nonwoven substrate. The nonwoven substrate of Test Sample 7 is a 27 gsm carded polypropylene. For reference, the circular shape is generally represented by a circular-shaped macro-unit shown in FIG. 8 as having a Upd of 7.5 mm and 6 color zones.

TABLE 7
L* Measurements from Test Sample 7

| L*₁ | L*₂ | L*₃ | L*₄ | L*₅ | L*₆ | ΔL*₁₂ | ΔL*₂₃ | ΔL*₃₄ | ΔL*₄₅ | ΔL*₅₆ |
|---|---|---|---|---|---|---|---|---|---|---|
| 94.4 | 91.7 | 88.6 | 85.9 | 82.2 | 79.3 | 2.7 | 3.1 | 2.7 | 3.7 | 2.9 |

### Test Sample 8

Test Sample 8 includes a circular-shaped macro-unit with a 7.5 mm diameter printed on a nonwoven-film laminate substrate and having six color zones, wherein the lightest color zone is defined by the color of the nonwoven-film substrate and the other five color zones are printed on the nonwoven-film substrate. Specifically, the macro-unit is printed onto a nonwoven fabric, which is adhered to a film substrate. The nonwoven-film substrate of Test Sample 8 is a 27 gsm carded polypropylene nonwoven adhered to a 18 gsm polypropylene/polyethylene (PP/PE) film. For reference, the circular shape is generally represented by a circular-shaped macro-unit shown in FIG. 8 as having a Upd of 7.5 mm and 6 color zones.

TABLE 8
L* Measurements from Test Sample 8

| L*₁ | L*₂ | L*₃ | L*₄ | L*₅ | L*₆ | ΔL*₁₂ | ΔL*₂₃ | ΔL*₃₄ | ΔL*₄₅ | ΔL*₅₆ |
|---|---|---|---|---|---|---|---|---|---|---|
| 91.4 | 85.8 | 79.3 | 74.5 | 69.9 | 65.3 | 5.6 | 6.5 | 4.8 | 4.6 | 4.6 |

### Test Sample 9

Test Sample 9 includes a circular-shaped macro-unit with a 7.5 mm diameter printed on a film substrate and having six color zones, wherein the lightest color zone is defined by the color of the film substrate and the other five color zones are printed on the film substrate. The film substrate of Test Sample 9 is a 18 gsm polypropylene/polyethylene (PP/PE) film. For reference, the circular shape is generally represented by a circular-shaped macro-unit shown in FIG. 8 as having a Upd of 7.5 mm and 6 color zones.

TABLE 9
L* Measurements from Test Sample 9

| L*₁ | L*₂ | L*₃ | L*₄ | L*₅ | L*₆ | ΔL*₁₂ | ΔL*₂₃ | ΔL*₃₄ | ΔL*₄₅ | ΔL*₅₆ |
|---|---|---|---|---|---|---|---|---|---|---|
| 97.5 | 94.4 | 89.4 | 83.0 | 75.4 | 68.8 | 3.1 | 5.0 | 7.0 | 7.6 | 6.6 |

As illustrated by the data in Tables 7-9, the macro-units of Test Samples 7, 8, and 9 have zones with L* values that fall within the following criteria described above:

L1>L2>L3>L4>L5,

2≦(L1−L2)≦10,

2≦(L2−L3),

2≦(L3−L4),

2≦(L4−L5); and

2≦(L5−L6).

### Test Sample 10

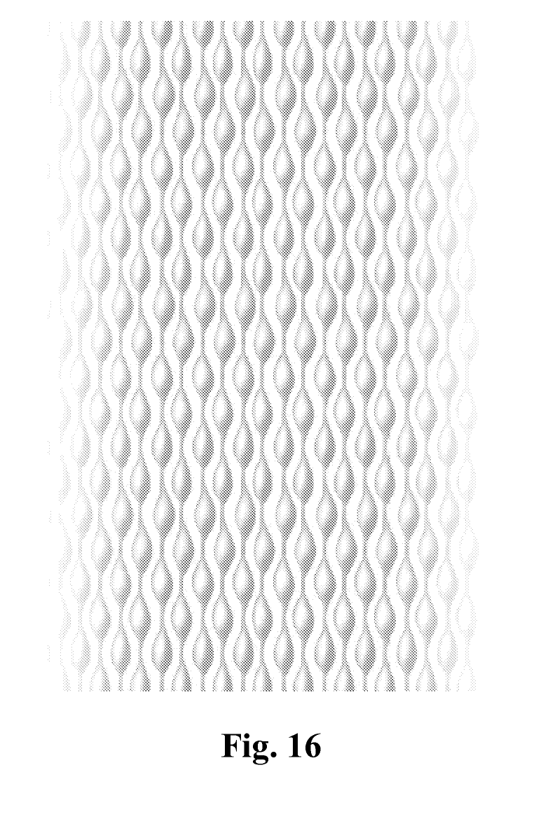

Test Sample 10 includes a repeating pattern of macro-units generally represented by the pattern shown in FIG. 16 printed on a nonwoven substrate and having three color zones, wherein the lightest color zone is defined by the color of the nonwoven substrate and the other two color zones are printed on the nonwoven substrate. The nonwoven substrate of Test Sample 10 is a 15 gsm spunbonded pure polypropylene.

TABLE 10
L* Measurements from Test Sample 10

| L*₁ | L*₂ | L*₃ | ΔL*₁₂ | ΔL*₂₃ | ΔL*₁₃ |
|---|---|---|---|---|---|
| 93.5 | 89.4 | 85.9 | 5.3 | 3.5 | 8.2 |

### Test Sample 11

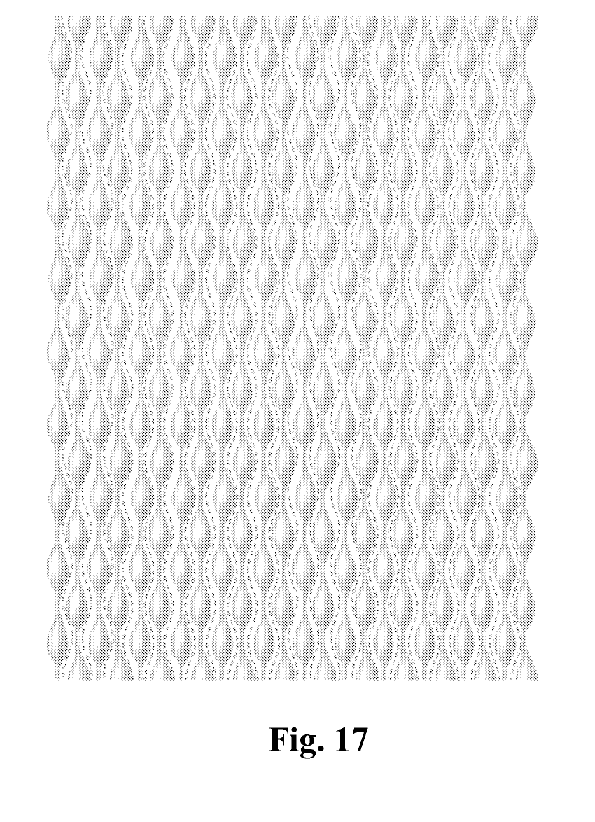

Test Sample 11 includes a repeating pattern of macro-units generally represented by the pattern shown in FIG. 17 printed on a nonwoven substrate and having three color zones, wherein the lightest color zone is defined by the color of the nonwoven substrate and the other two color zones are printed on the nonwoven substrate. The nonwoven substrate of Test Sample 11 is a 15 gsm spunbonded pure polypropylene.

TABLE 11
L* Measurements from Test Sample 11

| L*₁ | L*₂ | L*₃ | ΔL*₁₂ | ΔL*₂₃ | ΔL*₁₃ |
|---|---|---|---|---|---|
| 92.0 | 89.0 | 85.1 | 3.0 | 4.5 | 6.9 |

### Test Sample 12

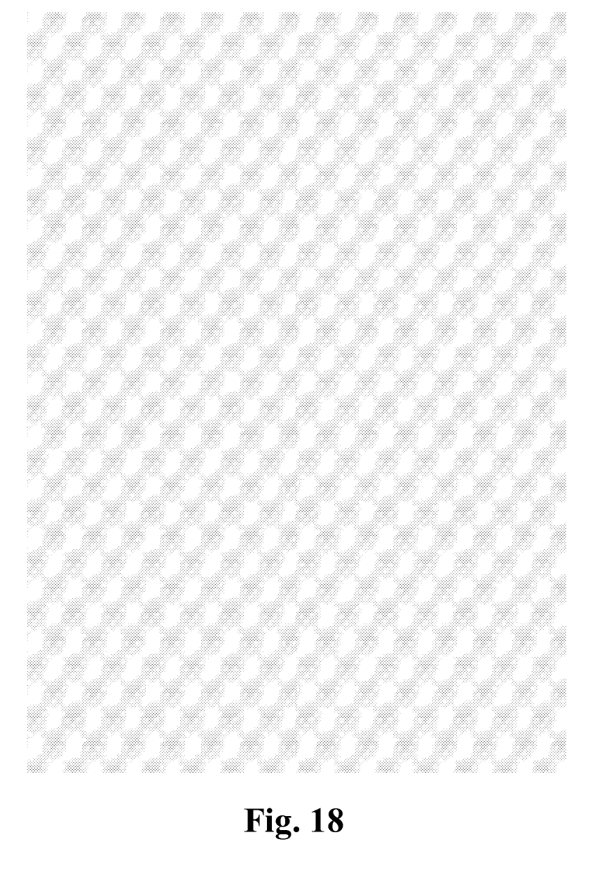

Test Sample 12 includes a repeating pattern of macro-units generally represented by the pattern shown in FIG. 18 printed on a film substrate and having three color zones, wherein the lightest color zone is defined by the color of the film substrate and the other two color zones are printed on the film substrate. The film substrate of Test Sample 12 is a 18 gsm polypropylene/polyethylene film.

TABLE 12
L* Measurements from Test Sample 12

| L*₁ | L*₂ | L*₃ | ΔL*₁₂ | ΔL*₂₃ | ΔL*₁₃ |
|---|---|---|---|---|---|
| 98.8 | 95.7 | 93.7 | 3.1 | 2.0 | 5.1 |

As illustrated by the data in Tables 10-12, the macro-units of Test Samples 10, 11, and 12 have zones with L* values that fall within the following criteria described above:

L1>L2>L3,

3≦(L1−L3), and

2≦(L1−L2)≦10.

### L* Measurement Procedure

Color measurements are performed using a commercial flat bed scanner capable of 4800 dpi, at 16 bit color depth, such as an Epson Perfection V500 Photo scanner (Epson America, Long Beach, Calif.). Each scan is calibrated against Pantone standards, and measurements made using Adobe Photoshop CS3 Extended Edition (Adobe Systems, Inc, San Jose, Calif.). The sample is always measured on the printed side of the substrate. For example, if a laminate consist of a nonwoven and a film where the printing is on the film and sandwiched between the film and nonwoven, the nonwoven is removed before the printing on the film is measured.

Scans are calibrated using the Pantone Process Colors standard from the Pantone Formula Guide—Uncoated Papers (Pantone, Carlstadt, N.J.). CIE L*a*b* values are measured for the Pantone standard for each color, i.e., Process Yellow U, Process Magenta U, Process Cyan U, Process Black U, and the White uncoated paper. Tristimulus colors were measured according to ASTM Method E1164-07 (Standard Practice for Obtaining Spectrophotometric Data for Object-Color Evaluation) using a Hunter Labscan XE (HunterLab, Reston, Va.) with HunterLab Universal Software vs. 4.10 with the following settings: Scale CIELAB, 0/45 StdMode, Area View 0.50 in., Port Size 0.70 in., UV filter Nominal. During measurement the standard is backed using the white calibration plate provided by HunterLab. Each color should be measured at least in triplicate and averaged.

The sample is placed on the scanner with the printed-side toward the sensor. The Pantone standard is also placed on the scanner such that the sample and standard are both captured in the same image.

The scan is collected at 1200 dpi at 8 bit color depth into Photoshop for objects with a primary dimension of greater than 3 mm, and at 2400 dpi, 8 bit color depth for objects with a primary dimension of less than 3 mm. Within Photoshop, the image is transformed into a Lab, 8 bit image (note in this version of Photoshop, L*a*b* is imprecisely denoted as Lab). Using the “Levels” command, the L channel of the image is adjusted to read within 2 units for each of the yellow, magenta, cyan, black and white colors on the Pantone standard. L*a*b* values are measured using the Color Sampler Tool using an 11 by 11 average sample size.

When measuring the sample, the printed object is first identified. Next the lightest zone (i.e., highest L value) is measured via the Color Sampler Tool. Then the darkest zone is measured via the Color Sampler Tool. Finally, measures are made at each intermediate zone between those two zones, along a linear path from the lightest to the darkest. At least one set of measurements on 10 distinct objects are made for each sample.

### Stiffness of Fabric Test

The Stiffness of Fabric Test is run for purpose of the present specification is a modification of the Stiffness of Fabric Test by Circular bend as described in the ASTM D 4032-94 which is hereby incorporated by reference. The Stiffness of Fabric Test for purposes of the present specification is conducted as follows:

### Summary of Test Method

A pusher-ball forces a swatch of material through an orifice in a platform. The maximum force required to push the fabric through the orifice is an indication of the material's stiffness (resistance to bending).

### Apparatus

Circular Bend Stiffness Tester, having the following parts:
Platform, 102 mm×102 mm×6 mm smooth-polished chrome-plated steel plate with a 38.1-mm diameter orifice. The lap edge of the orifice should be at a 45° angle to a depth of 4.8 mm.
Pusher-Ball, 6 mm diameter steel spherical ball, mounted concentric with orifice, 16 mm clearance on all sides. The bottom of the pusher-ball plunger should be set at 3 mm above the top of the orifice plate. From this position, the downward stroke length is 57 mm.
Force-Measurement Gauge, dial or digital type dial gauges with maximum reading pointer in different capacities ranging from 1 to 50 lbf, 0.5 to 25 kgf, or 5 to 200 N with 100 graduations minimum; or digital gauge with maximum reading “hold” feature and capacity of 100 lbf, 50 kgf, or 500 N, with 1000 graduations minimum.
Actuator, manual or pneumatic.
Specimen Marking Template, 102 mm×102 mm.
Stop Watch, for checking stroke speed.

### Number and Preparation of Test Specimens

Using the specimen marking template specified above mark and cut five test specimens from staggered areas of each swatch of material to be tested. It will be appreciated that it may not be practical or possible to obtain all samples from a particular swatch (or particular product if the material is only available as incorporated into a product). In such a case, it is acceptable to take samples from multiple products or swatches. Samples with bonded, seals, seams or the like should be avoided. Lay each specimen flat to form a square 102 mm×102 mm. Handling of specimens should be kept to a minimum and to the edges to avoid affecting stiffness properties.

### Conditioning

Store the samples for 8 hours or more at 23° C. and 50% relative humidity.

### Procedure

Set the tester on a flat surface with dial at eye level.
Select a gage with a capacity in which results will fall within 15 to 100% of dial gage force or 1.5 to 100% of digital gage force.
Check tester pusher-ball speed control for full stroke length.
Pneumatic Actuator—Set the air pressure control to the actuator at 324 kPa. Using a stop-watch, adjust the pneumatics to provide plunger speed of 1.7±0.15 s under no load conditions.
Manual Actuator—Using a stop-watch, establish and confirm a plunger speed of 1.7±0.3 s.
Center a specimen on the orifice platform below the pusher-ball.
If 3.2 mm clearance under pusher-ball prevents ease of entry of specimen due to sample thickness, the clearance may be increased to 6.3 mm maximum. In reporting, the results should indicate the pusher-ball clearance, if not standard.
Check the gage zero and adjust, if necessary.
Set the maximum force reading switch.
Actuate the pusher-ball for the full stroke length. Avoid touching the specimen during testing.
Record maximum force reading to nearest gage graduation.
Continue as directed above until all specimens have been tested.

### Calculation

Average the individual specimen readings and round to the nearest gage increment.

### Report

Report the Average force in gage units.

End of Stiffness Fabric Test

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article adapted to be worn about a lower torso region of a wearer comprising:
a chassis comprising: a topsheet, a backsheet, and an absorbent core disposed between the topsheet and the backsheet, the topsheet and the backsheet defining a first waist region longitudinally opposed to a second waist region, wherein the first and second waist regions are connectable with each other to form a waist opening;
wherein the backsheet includes a body facing surface and a garment facing surface;
a repeating pattern of macro-units printed directly on the garment facing surface of the backsheet, the repeating pattern of macro-units resembling protrusions and indentions indicative of threads in woven cloths;
wherein the macro-units include a first color zone defining a L* value of L1, a second color zone defining a L* value of L2, and a third color zone defining a L* value of L3; and
wherein L1>L2>L3, 3≦(L1−L3), and 2≦(L1−L2)≦10.

2. The disposable absorbent article of claim 1, wherein the backsheet comprises a film material and a nonwoven material, the film material having a body facing surface and a garment facing surface, and wherein the nowoven material is joined with the garment facing surface of the film material; and
wherein the repeating pattern of macro-units is printed directly on the garment facing surface of the film material.

3. The disposable absorbent article of claim 1, wherein the backsheet comprises a film material and a nonwoven material, the film material having a body facing surface and a garment facing surface, and wherein the nonwoven material is joined with the garment facing surface of the film material; and
wherein the repeating pattern of macro-units is printed directly on the nonwoven material.

4. The disposable absorbent article of claim 1, wherein the backsheet comprises a nonwoven material having a body facing surface and a garment facing surface; and
wherein the repeating pattern of macro-units is printed directly on the nonwoven material.

5. The disposable absorbent article of claim 1, wherein each macro-unit includes a first lateral print point and a second lateral print point separated by a distance of Dlat, and wherein each macro-unit includes a first longitudinal point and a second longitudinal point separated by a distance of Dlong, and wherein the macro-unit includes a primary dimension, Upd, defined by the minimum of Dlong and Dlat, wherein Upd is greater than or equal to 1.5 mm.

6. The disposable absorbent article of claim 1, wherein the repeating pattern of macro-units defines an outer perimeter wherein a smallest theoretical square or rectangle can surround the outer perimeter;
wherein each macro-unit can be surrounded by a print point rectangle or square; and
wherein the maximum distance between print point rectangles or squares of adjacent macro-units is defined by 0.1×(a length of a longest side of the smallest theoretical square or rectangle).

7. The disposable absorbent article of claim 1, wherein the repeating pattern of macro-units are flexographically printed.

8. The disposable absorbent article of claim 1, wherein the repeating pattern of macro-units are inkjet printed.

9. A disposable absorbent article adapted to be worn about a lower torso region of a wearer comprising:
a chassis comprising: a topsheet, a backsheet, and an absorbent core disposed between the topsheet and the backsheet, the topsheet and the backsheet defining a first waist region longitudinally opposed to a second waist region, wherein the first and second waist regions are connectable with each other to form a waist opening;
wherein the backsheet includes a body facing surface and a garment facing surface;
a repeating pattern of macro-units printed directly on the garment facing surface of the backsheet, the repeating pattern of macro-units resembling protrusions and indentions indicative of threads in woven cloths;
wherein the macro-units include at least a first color zone defining a L* value of L1, a second color zone defining a L* value of L2, a third color zone defining a L* value of L3, and a fourth color zone defining a L* value of L4; and
wherein L1>L2>L3>L4, 2≦(L1−L2)≦10, 2≦(L2−L3), and 2≦(L3−L4).

10. The disposable absorbent article of claim 9, wherein the backsheet comprises a film material having a body facing surface and a garment facing surface; and
wherein the repeating pattern of macro-units is printed directly on the garment facing surface of the film material.

11. The disposable absorbent article of claim 10, wherein the backsheet further comprises a nonwoven material joined with the garment facing surface of the film material.

12. The disposable absorbent article of claim 9, wherein the backsheet comprises a film material and a nonwoven material, the film material having a body facing surface and a garment facing surface, and wherein the nonwoven material is joined with the garment facing surface of the film material; and
wherein the repeating pattern of macro-units is printed directly on the nonwoven material.

13. The disposable absorbent article of claim 9, wherein the backsheet comprises a nonwoven material having a body facing surface and a garment facing surface; and
wherein the repeating pattern of macro-units is printed directly on the nonwoven material.

14. The disposable absorbent article of claim 9, wherein each macro-unit includes a first lateral print point and a second lateral print point separated by a distance of Dlat, and wherein each macro-unit includes a first longitudinal point and a second longitudinal point separated by a distance of Dlong, and wherein the macro-unit includes a primary dimension, Upd, defined by the minimum of Dlong and Dlat, wherein Upd is greater than or equal to 1.5 mm.

15. The disposable absorbent article of claim 9, wherein the repeating pattern of macro-units defines an outer perimeter wherein a smallest theoretical square or rectangle can surround the outer perimeter;
wherein each macro-unit can be surrounded by a print point rectangle or square; and
wherein the maximum distance between print point rectangles or squares of adjacent macro-units is defined by 0.1×(a length of a longest side of the smallest theoretical square or rectangle).

16. The disposable absorbent article of claim 9, wherein the repeating pattern of macro-units are flexographically printed.

17. The disposable absorbent article of claim 9, wherein the repeating pattern of macro-units are inkjet printed.
